(19) United States
(12) Patent Application Publication
(10) Pub. No.: US 2024/0161543 A1
(43) Pub. Date: May 16, 2024

(54) BIOLOGICAL INFORMATION PROCESSING DEVICE AND BIOLOGICAL INFORMATION PROCESSING SYSTEM

(71) Applicant: SONY GROUP CORPORATION, TOKYO (JP)

(72) Inventor: NAOYA SAZUKA, TOKYO (JP)

(21) Appl. No.: 18/550,979

(22) PCT Filed: Feb. 25, 2022
(86) PCT No.: PCT/JP2022/008062

§ 371 Date: Sep. 18, 2023

(30) Foreign Application Priority Data
Mar. 29, 2021 (JP) 2021-056031
Aug. 17, 2021 (JP) 2021-132937

(51) Int. Cl. G06V 40/16; A61B 5/16; G06N 3/04

(52) U.S. Cl. CPC G06V 40/174; A61B 5/16; G06N 3/04; G06V 40/172

(57) ABSTRACT

A biological information processing device according to an aspect of the present disclosure includes a derivation section and a classification section. The derivation section derives emotional information about a target living body who is performing a specific task on the basis of at least one of biological information or action information acquired from the target living body. The classification section classifies the emotional information acquired by the derivation section on the basis of a predetermined classification index.

### TECHNICAL FIELD

The present disclosure relates to a biological information processing device and a biological information processing system.

### BACKGROUND ART

For any organization, securing talented human resources is necessary for prosperity of the organization. However, it is difficult to find talented human resources. In order to perform recruitment activity or selection of a person for team building in an organization, human intuition, experiences, and subjectivity have been often used, or attribute information such as age, gender, and educational background has been often used. Examples of existing technology for evaluating a person by attributes include the following PTL 1 and the like.

### CITATION LIST

### Patent Literature

PTL 1: Japanese Unexamined Patent Application Publication No. 2019-101720

### SUMMARY OF THE INVENTION

In a case where human intuition, experiences, and subjectivity are used, an interviewer subjectively makes a decision by a short-time interview or the like. Such short-time subjective confirmation may cause a recruitment mismatch resulting from an oversight or a case where the interviewer's field of specialization is different from the field of specialization of a wanted talent. In addition, even in a case where a team is assembled in an organization to manage a project, members may be determined only by superficial specialization or superior's subjectivity. This may cause a mismatch similar to that described above.

Even in a case where attribute information such as age, gender, and educational background is used, objective abilities of individuals are not reflected in determination, which may cause an opportunity loss such as a possibility that determination is uniformly made by age or the like. In addition, such a mismatch may be caused not only in recruitment and selection of project members but also in various types of selection of living bodies other than humans. It is therefore desirable to provide a biological information processing device and a biological information processing system that make it possible to reduce mismatches.

A biological information processing device according to a first aspect of the present disclosure includes a derivation section and a classification section. The derivation section derives emotional information about a target living body who is performing a specific task on the basis of at least one of biological information or action information acquired from the target living body. The classification section classifies the emotional information acquired by the derivation section on the basis of a predetermined classification index.

A biological information processing system according to a second aspect of the present disclosure includes an acquisition section, a derivation section, and a classification section. The acquisition section acquires at least one of biological information or action information from a target living body who is performing a specific task. The derivation section derives emotional information about the target living body on the basis of information acquired by the acquisition section. The classification section classifies the emotional information acquired by the derivation section on the basis of a predetermined classification index.

In the biological information processing device according to the first aspect of the present disclosure and the biological information processing system according to the second aspect of the present disclosure, the emotional information is classified on the basis of the predetermined classification index. This makes it possible to classify the target living body with use of the emotional information that is objective data. As a result, for example, in a case of recruitment, it is possible to determine, from emotional information about an applicant, whether or not the applicant is a desired talent. In addition, for example, in a case of determining project members, it is possible to determined, from emotional information about a large number of members, whether or not the members are suitable to constitute the specific group.

A biological information processing device according to a third aspect of the present disclosure includes a storage section and a derivation section. The derivation section derives emotional information about a target living body who is performing a specific task on the basis of at least one of biological information or action information acquired from the target living body. Furthermore, the derivation section stores, in the storage section, the derived emotional information in association with an identifier of the target living body.

A biological information processing system according to a fourth aspect of the present disclosure includes a storage section, an acquisition section, and a derivation section. The acquisition section acquires at least one of biological information or action information from a target living body who is performing a specific task. The derivation section derives emotional information about the target living body on the basis of the information acquired by the acquisition section. Furthermore, the derivation section stores, in the storage section, the derived emotional information in association with an identifier of the target living body.

In the biological information processing device according to the third aspect of the present disclosure and the biological information processing system according to the fourth aspect of the present disclosure, the derived emotional information is stored, in the storage section, in association with the identifier of the target living body. This makes it possible to classify the target living body with use of the arousal level that is objective data. Aa a result, for example, in a case of recruitment, it is possible to determine, from the arousal level of an applicant, whether or not the applicant is a desired talent. In addition, for example, in a case of determining project members, it is possible to determine, from emotional information about a large number of members, whether or not the members are suitable to constitute the specific group.

### BRIEF DESCRIPTION OF DRAWINGS

FIG. 1 is a diagram illustrating an example of a schematic configuration of a biological information processing system according to a first embodiment of the present disclosure.

FIG. 2 is a diagram illustrating an example of functional blocks of an electronic apparatus in FIG. 1.

FIG. 3 is a diagram illustrating an example of a relationship between task processing time and an arousal level.

FIG. 4 is a diagram of a relationship between duration and rise time that is classified into four.

FIG. 5 is a diagram illustrating an example of a display screen.

FIG. 6 is a diagram illustrating an example of the display screen.

FIG. 7 is a diagram illustrating an example of the display screen.

FIG. 8 is a diagram illustrating an example of a processing procedure in the biological information processing system in FIG. 1.

FIG. 9 is a diagram illustrating an example of a schematic configuration of a biological information processing system according to a second embodiment of the present disclosure.

FIG. 10 is a diagram illustrating an example of functional blocks of an electronic apparatus in FIG. 9.

FIG. 11 is a diagram illustrating an example of a processing procedure in the biological information processing system in FIG. 9.

FIG. 12 is a diagram illustrating an example of a schematic configuration of an information processing system according to a third embodiment of the present disclosure.

FIG. 13 is a diagram illustrating an example of functional blocks of an electronic apparatus in FIG. 12.

FIG. 14 is a diagram illustrating an example of functional blocks of the electronic apparatus in FIG. 12.

FIG. 15 is a diagram illustrating an example of a processing procedure in the information processing system in FIG. 12.

FIG. 16 is a diagram illustrating an example of a schematic configuration of an information processing device according to a fourth embodiment of the present disclosure.

FIG. 17 is a diagram illustrating an example using an estimation model in the biological information processing systems in FIGS. 1 and 9, the information processing system in FIG. 12, and the information processing device in FIG. 16.

FIG. 18 is a diagram illustrating an example using attribute information in the biological information processing systems in FIGS. 1 and 9, the information processing system in FIG. 12, and the information processing device in FIG. 16.

FIG. 19 is a diagram illustrating an example of time series data of reaction times for low difficulty level questions.

FIG. 20 is a diagram illustrating an example of time series data of reaction times for high difficulty level questions.

FIG. 21 is a diagram illustrating an example of a power spectrum density obtained by performing FFT (Fast Fourier Transform) on observation data of a brain wave (α-wave) of a user when solving low difficulty level questions.

FIG. 22 is a diagram illustrating an example of a power spectrum density obtained by performing FFT (Fast Fourier Transform) on observation data of a brain wave (α-wave) of the user when solving high difficulty level questions.

FIG. 23 is a diagram illustrating an example of a relationship between a task difference in dispersion of reaction times and a task difference in a peak value of power of a brain wave in a low-frequency band.

FIG. 24 is a diagram illustrating an example of a relationship between a task difference in dispersion of reaction times and a task difference in an accuracy rate.

FIG. 25 is a diagram illustrating an example of a relationship between a task difference in an arousal level and a task difference in the peak value of the power of the brain wave in the low-frequency band.

FIG. 26 is a diagram illustrating an example of a relationship between the task difference in the arousal level and the task difference in the accuracy rate.

FIG. 27 is a diagram illustrating an example of a relationship between the dispersion of the reaction times and the accuracy rate.

FIG. 28 is a diagram illustrating an example of a relationship between the arousal level and the accuracy rate.

FIG. 29 is a diagram illustrating an example of a head-mounted display mounted with a sensor.

FIG. 30 is a diagram illustrating an example of a head band mounted with a sensor.

FIG. 31 is a diagram illustrating an example of a headphone mounted with a sensor.

FIG. 32 is a diagram illustrating an example of an earphone mounted with a sensor.

FIG. 33 is a diagram illustrating an example of a watch mounted with a sensor.

FIG. 34 is a diagram illustrating an example of glasses mounted with a sensor.

FIG. 35 is a diagram illustrating an example of a relationship between a task difference in pnn50 of a pulse wave and the accuracy rate.

FIG. 36 is a diagram illustrating an example of a relationship between a task difference in dispersion of the pnn50 of the pulse wave and the accuracy rate.

FIG. 37 is a diagram illustrating an example of a relationship between a task difference in power of the pnn50 of the pulse wave in the low-frequency band and the accuracy rate.

FIG. 38 is a diagram illustrating an example of a relationship between a task difference in rmssd of the pulse wave and the accuracy rate.

FIG. 39 is a diagram illustrating an example of a task difference in dispersion of the rmssd of the pulse wave and the accuracy rate.

FIG. 40 is a diagram illustrating an example of a relationship between a task difference in power of the rmssd of the pulse wave in the low-frequency band and the accuracy rate.

FIG. 41 is a diagram illustrating an example of a relationship between a task difference in dispersion of the number of SCRs of emotional sweating and the accuracy rate.

FIG. 42 is a diagram illustrating an example of a relationship between a task difference in the number of SCRs of the emotional sweating and the accuracy rate.

FIG. 43 is a diagram illustrating an example of a relationship between a task difference in a median value of reaction times and the accuracy rate.

FIG. 44 is a diagram illustrating an example of a relationship between the arousal level and the accuracy rate.

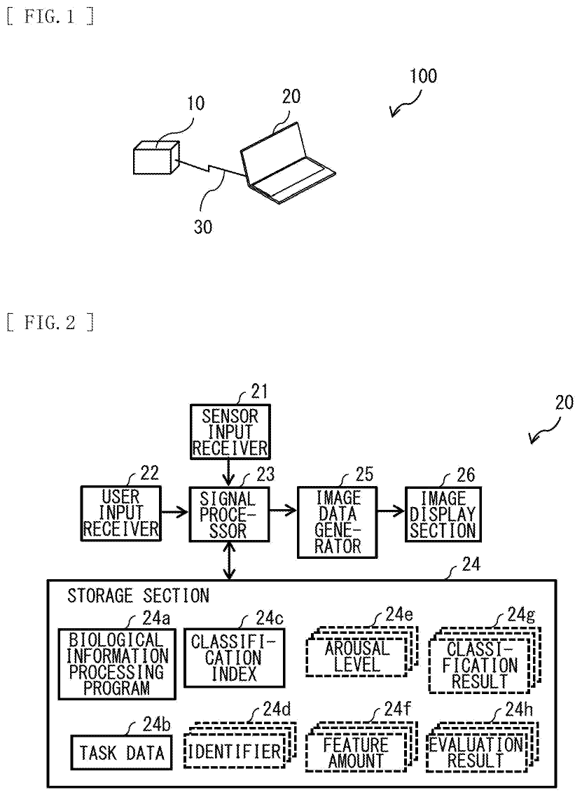

### MODES FOR CARRYING OUT THE INVENTION

In the following, some embodiments of the present disclosure are described in detail with reference to the drawings.

<1. About Arousal Level>

The arousal level of a person is closely related to concentration of the person. A person in concentration exhibits a high ability. Accordingly, it is possible to estimate an objective ability of the person by knowing the arousal level of the person. It is possible to derive the arousal level of the person on the basis of biological information or action information acquired from the person who is performing a specific task (hereinafter referred to as a “target living body”).

Examples of the biological information from which an arousal level of the target living body is derivable include information about a brain wave, sweating, a pulse wave, an electrocardiogram, a blood flow, a skin temperature, potential of facial muscles, eye potential, a specific component contained in saliva.

(Brain Wave)

It is known that α-waves included in brain waves increase when a person is relaxed such as when resting, and β-waves included in brain waves increase when the person is doing actively active thinking or is in concentration. Accordingly, for example, when a power spectrum area in a frequency band of the α-wave included in the brain wave is smaller than a predetermined threshold th1 and a power spectrum area in a frequency band of the β-wave included in the brain wave is larger than a predetermined threshold th2, it is possible to estimate that the arousal level of the target living body is high.

In addition, in estimating the arousal level of the target living body with use of the brain wave, it is possible to use an estimation model such as machine learning in place of the thresholds th1 and th2. This estimation model is, for example, a model trained using, as teaching data, a power spectrum of the brain wave when the arousal level is apparently high. For example, in a case where a power spectrum of the brain wave is inputted, this estimation model estimates the arousal level of the target living body on the basis of the inputted power spectrum of the brain wave. This estimation model includes, for example, a neural network. This learning model may include, for example, a deep neural network such as a convolutional neural network (CNN).

In addition, the brain wave may be divided into a plurality of segments on a time axis, and a power spectrum may be derived for each of divided segments to derive a power spectrum area in the frequency band of the α-wave for each derived power spectrum. In this case, for example, when the derived power spectrum area is smaller than a predetermined threshold tha, it is possible to estimate that the arousal level of the target living body is high.

In addition, it is possible to estimate the arousal level of the target living body with use of, for example, an estimation model that estimates the arousal level of the target living body on the basis of the derived power spectrum area. This estimation model is, for example, a model trained using, as teaching data, a power spectrum area when the arousal level is apparently high. For example, in a case where a power spectrum area is inputted, this estimation model estimates the arousal level of the target living body on the basis of the inputted power spectrum area. This estimation model includes, for example, a neural network. This learning model may include, for example, a deep neural network such as a convolutional neural network (CNN).

(Sweating)

Emotional sweating is sweating released from eccrine sweat glands when sympathetic nerves are strained due to a mental/psychological issue such as stress, tension, or anxiety. For example, a sweat sensor probe is attached to a palm or a sole, and sweating (emotional sweating) on the palm or the sole induced by various load stimuli is measured, which makes it possible to obtain a sympathetic sweat response (SSwR) as a signal voltage. When a numeric value of a predetermined high-frequency component or a predetermined low-frequency component in this signal voltage is higher than a predetermined threshold, it is possible to estimate that the arousal level of the target living body is high.

In addition, it is possible to estimate the arousal level of the target living body with use of, for example, an estimation model that estimates the arousal level of the target living body on the basis of the predetermined high-frequency component or the predetermined low-frequency component included in this signal voltage. This estimation model is, for example, a model trained using, as teaching data, a predetermined high-frequency component or a predetermined low-frequency component included in a signal voltage when the arousal level is apparently high. For example, in a case where the predetermined high-frequency component or the predetermined low-frequency component is inputted, this estimation model estimates the arousal level of the target living body on the basis of the inputted predetermined high-frequency component or the inputted predetermined low-frequency component. This estimation model includes, for example, a neural network. This learning model may include, for example, a deep neural network such as a convolutional neural network (CNN).

(Pulse Wave, Electrocardiogram, and Blood Flow)

It is generally said that, when a heart rate is high, the arousal level is high. It is possible to derive the heart rate from a pulse wave, an electrocardiogram, or blood flow velocity. Accordingly, for example, when the heart rate is derived from the pulse wave, the electrocardiogram, or the blood flow velocity and the derived heart rate is larger than a predetermined threshold, it is possible to estimate that the arousal level of the target living body is high.

In addition, it is possible to estimate the arousal level of the target living body with use of, for example, an estimation model that estimates the arousal level of the target living body on the basis of the heart rate derived from the pulse wave, the electrocardiogram, or the blood flow velocity. This estimation model is, for example, a model trained using, as teaching data, a heart rate when the arousal level is apparently high. In a case where a heart rate derived from the pulse wave, the electrocardiogram, or the blood flow velocity is inputted, this estimation model estimates the arousal level of the target living body on the basis of the inputted heart rate. This estimation model includes, for example, a neural network. This learning model may include, for example, a deep neural network such as a convolutional neural network (CNN).

In addition, it is generally said that, when heart rate variability (HRV) is small, parasympathetic nerves become subordinate and the arousal level is high. Accordingly, for example, when the heart rate variability (HRV) is derived from the pulse wave, the electrocardiogram, or the blood flow velocity and the derived heart rate variability (HRV) is smaller than a predetermined threshold, it is possible to estimate that the arousal level of the target living body is high.

In addition, it is possible to estimate the arousal level of the target living body with use of, for example, an estimation model that estimates the arousal level of the target living body on the basis of the heart rate variability (HRV) derived from the pulse wave, the electrocardiogram, or the blood flow velocity. This estimation model is, for example, a model trained using, as teaching data, heart rate variability (HRV) when the arousal level is apparently high. For example, in a case where heart rate variability (HRV) derived from the pulse wave, the electrocardiogram, or the blood flow velocity is inputted, this estimation model estimates the arousal level of the target living body on the basis of the inputted heart rate variability (HRV). This estimation model includes, for example, a neural network. This learning model may include, for example, a deep neural network such as a convolutional neural network (CNN).

(Skin Temperature)

It is generally said that, when a skin temperature is high, the arousal level is high. It is possible to measure the skin temperature by, for example, thermography. Accordingly, for example, when the skin temperature measured by the thermography is higher than a predetermined threshold, it is possible to estimate that the arousal level of the target living body is high.

In addition, it is possible to estimate the arousal level of the target living body with of, for example, an estimation model that estimates the arousal level of the target living body on the basis of the skin temperature. This estimation model is, for example, a model trained using, as teaching data, a skin temperature when the arousal level is apparently high. For example, in a case where a skin temperature is inputted, this estimation model estimates the arousal level of the target living body on the basis of the inputted skin temperature. This estimation model includes, for example, a neural network. This learning model may include, for example, a deep neural network such as a convolutional neural network (CNN).

(Potential of Facial Muscles)

It is known that corrugator supercilii muscles involved in frowning show high activity when thinking about something. In addition, it is known that zygomaticus major muscles hardly change when thinking about happy things. Thus, it is possible to estimate emotion or the arousal level in accordance with facial parts. Accordingly, for example, when potential of facial muscles in a predetermined part is measured and a thus-obtained measurement value is higher than a predetermined threshold, it is possible to estimate whether the arousal level of the target living body is high or low.

In addition, it is possible to estimate the arousal level of the target living body with use of, for example, an estimation model that estimates the arousal level of the target living body on the basis of the potential of facial muscles. This estimation model is, for example, a model trained using, as teaching data, potential of facial muscles when the arousal level is apparently high. For example, in a case where potential of facial muscles is inputted, this estimation model estimates the arousal level of the target living body on the basis of the inputted potential of facial muscles. This estimation model includes, for example, a neural network. This learning model may include, for example, a deep neural network such as a convolutional neural network (CNN).

(Eye Potential)

A method is known of measuring eyeball movement with use of a positively charged cornea and a negatively charged retina in a eyeball. A measurement value obtained by using this measuring method is an electrooculogram. For example, in a case where the eyeball movement is estimated from an obtained electrooculogram and the estimated eyeball movement has a predetermined tendency, it is possible to estimate whether the arousal level of the target living body is high or low.

In addition, it is possible to estimate the arousal level of the target living body with use of, for example, an estimation model that estimates the arousal level of the target living body on the basis of the electrooculogram. This estimation model is, for example, a model trained using, as teaching data, an electrooculogram when the arousal level is apparently high. For example, in a case where an electrooculogram is inputted, this estimation model estimates the arousal level of the target living body on the basis of the inputted electrooculogram. This estimation model includes, for example, a neural network. This learning model may include, for example, a deep neural network such as a convolutional neural network (CNN).

(Saliva)

Saliva contains cortisol that is a kind of stress hormone. It is known that, when getting stress, a cortisol content in the saliva increases. Accordingly, for example, when the cortisol content in saliva is higher than a predetermined threshold, it is possible to estimate that the arousal level of the target living body is high.

In addition, it is possible to estimate the arousal level of the target living body with use of, for example, an estimation model that estimates the arousal level of the target living body on the basis of the cortisol content in saliva This estimation model is, for example, a model trained using, as teaching data, a cortisol content in saliva when the arousal level is apparently high. For example, in a case where a cortisol content in saliva is inputted, this estimation model estimates the arousal level of the target living body on the basis of the inputted cortisol content. This estimation model includes, for example, a neural network. This learning model may include, for example, a deep neural network such as a convolutional neural network (CNN).

Meanwhile, examples of action information from which the arousal level of the target living body is derivable include information about facial expression, voice, blinking, breathing, or a reaction time for an action.

(Facial Expression)

It is known that eyebrows are knitted when thinking about something, and that zygomaticus major muscles hardly change when thinking about happy things. Thus, it is possible to estimate emotion or the arousal level in accordance with facial expression. Accordingly, for example, a face is photographed by a camera to estimate facial expression on the basis of thus-acquired moving image data, and it is possible to estimate whether the arousal level of the target living body is high or low, in accordance with the facial expression obtained by the estimation.

In addition, it is possible to estimate the arousal level of the target living body with use of, for example, an estimation model that estimates the arousal level of the target living body on the basis of moving image data acquired by photographing facial expression. This estimation model is, for example, a model trained using, as teaching data, moving image data acquired by photographing facial expression when the arousal level is apparently high. For example, in a case where moving image data acquired by photographing facial expression is inputted, this estimation model estimates the arousal level of the target living body on the basis of the inputted moving image data. This estimation model includes, for example, a neural network. This learning model may include, for example, a deep neural network such as a convolutional neural network (CNN).

(Voice)

It is known that voice changes in accordance with emotion or the arousal level similarly to facial expression. Accordingly, for example, voice data is acquired by a microphone, and it is possible to estimate whether the arousal level of the target living body is high or low, on the basis of the thus-acquired voice data.

In addition, it is possible to estimate the arousal level of the target living body with use of, for example, an estimation model that estimates the arousal level of the target living body on the basis of the voice data. This estimation model is, for example, a model trained using, as teaching data, voice data when the arousal level is apparently high. For example, in a case where voice data is inputted, this estimation model estimates the arousal level of the target living body on the basis of the inputted voice data. This estimation model includes, for example, a neural network. This learning model may include, for example, a deep neural network such as a convolutional neural network (CNN).

(Blinking)

It is known that blinking changes in accordance with emotion or the arousal level similarly to facial expression. Accordingly, for example, blinking is photographed by a camera to measure a frequency of blinking on the basis of thus-acquired moving image data, and it is possible to estimate whether the arousal level of the target living body is high or low, on the basis of the frequency of blinking obtained by the measurement. In addition, for example, the frequency of blinking is measured by an electrooculogram, and it is possible to estimate whether the arousal level of the target living body is high or low, in accordance with the frequency of blinking obtained by the measurement.

In addition, it is possible to estimate the arousal level of the target living body with use of, for example, an estimation model that estimates the arousal level of the target living body on the basis of moving image data acquired by photographing blinking or the electrooculogram. This estimation model is, for example, a model trained using, as teaching data, moving image data acquired by photographing blinking or an electrooculogram when the arousal level is apparently high. For example, in a case where moving image data acquired by photographing blinking or an electrooculogram is inputted, this estimation model estimates the arousal level of the target living body on the basis of the inputted moving image data or the inputted electrooculogram. This estimation model includes, for example, a neural network. This learning model may include, for example, a deep neural network such as a convolutional neural network (CNN).

(Breathing)

It is known that breathing changes in accordance with emotion or the arousal level similarly to facial expression. Accordingly, for example, a respiratory volume or a respiration rate is measured, and it is possible to estimate whether the arousal level of the target living body is high or low, on the basis of thus-acquired measurement data.

In addition, it is possible to estimate the arousal level of the target living body with use of, for example, an estimation model that estimates the arousal level of the target living body on the basis of the respiratory volume or the respiration rate. This estimation model is, for example, a model trained using, as teaching data, a respiratory volume or a respiration rate when the arousal level is apparently high. For example, in a case where a respiratory volume or a respiration rate is inputted, this estimation model estimates the arousal level of the target living body on the basis of the inputted respiratory volume or the inputted respiration rate. This estimation model includes, for example, a neural network. This learning model may include, for example, a deep neural network such as a convolutional neural network (CNN).

(Reaction Time for Action)

It is known that a processing time (reaction time) when a person processes a plurality of tasks in succession and dispersion of processing times (reaction times) depend on the arousal level of the person. Accordingly, for example, the processing time (reaction time) or the dispersion of the processing times (reaction times) is measured, and it is possible to estimate whether the arousal level of the target living body is high or low, on the basis of thus-acquired measurement data.

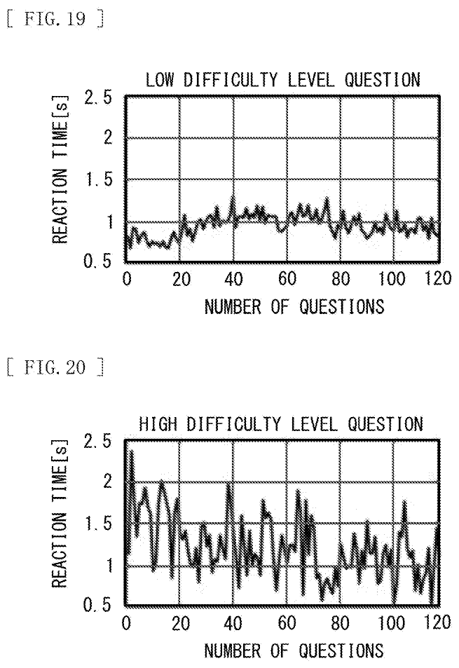

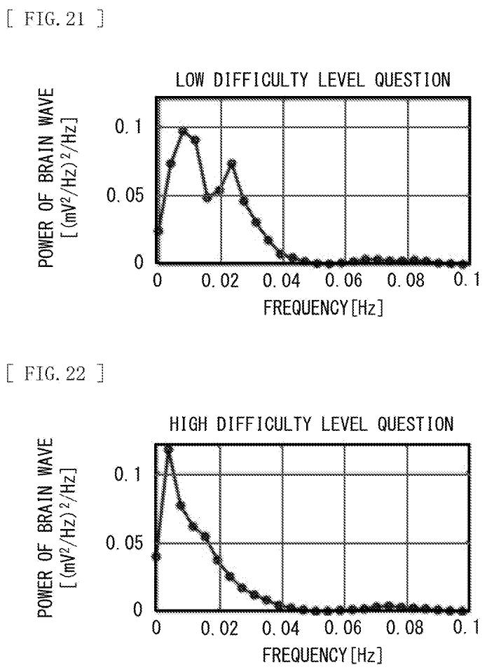

FIGS. 19 and 20 each illustrate, by way of a graph, time (reaction time) taken by a user to answer when the user solves a large number of questions in succession. FIG. 19 illustrates a graph at the time of solving questions of a relatively low difficulty level, and FIG. 20 illustrates a graph at the time of solving questions of a relatively high difficulty level. FIG. 21 illustrates a power spectrum density obtained by performing FFT (Fast Fourier Transform) on observation data of a brain wave (α-wave) of the user when the user solves a large number of low difficulty level questions in succession. FIG. 22 illustrates a power spectrum density obtained by performing FFT on observation data of a brain wave (α-wave) of the user when the user solves a large number of high difficulty level questions in succession. FIGS. 21 and 22 each illustrate a graph obtained by measuring a brain wave (α-wave) at a segment of about 20 seconds and performing FFT using an analysis window of about 200 seconds.

It is appreciated from FIGS. 19 and 20 that not only reaction time becomes longer, but also dispersion of the reaction times becomes larger at the time of solving high difficulty level questions, as compared with the time of solving low difficulty level questions. It is appreciated from FIGS. 21 and 22 that power of a brain wave (α-wave) around 0.01 Hz is larger and the power of a brain wave (α-wave) around 0.02 to 0.04 is smaller at the time of solving the high difficulty level questions, as compared with the time of solving the low difficulty level questions. As used herein, the power of the brain wave (α-wave) around 0.01 Hz is appropriately referred to as a “fluctuation in a slow (low-frequency band) brain wave (α-wave)”.

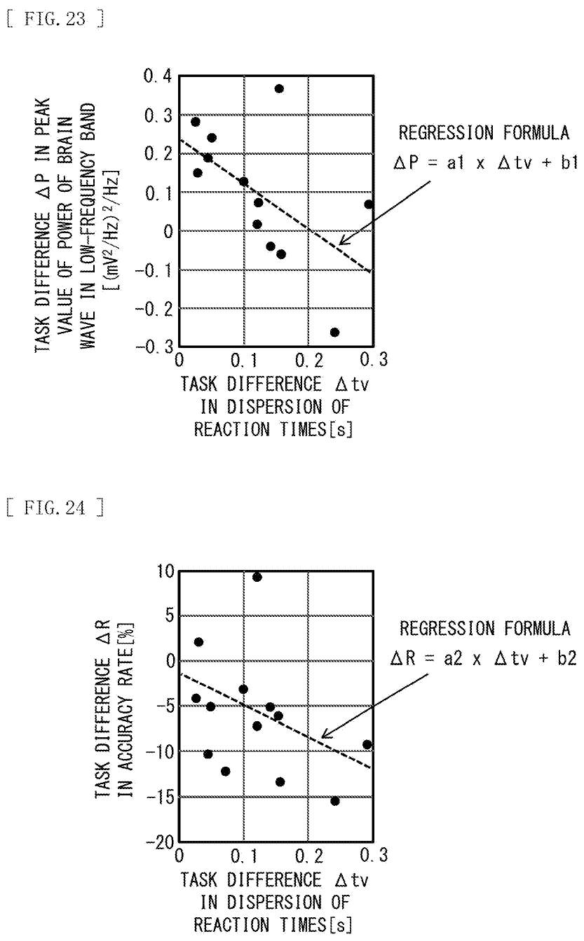

FIG. 23 illustrates an example of a relationship between a task difference Δtv [s] and a task difference ΔP [mV²/Hz)²/Hz]. The task difference Δtv [s] is a task difference in dispersion (75% percentile-25% percentile) of reaction times of the user between the time of solving the high difficulty level questions and the time of solving the low difficulty level questions. The task difference ΔP [mV²/Hz)²/Hz] is a task difference in a peak value of power of the slow brain wave (α-wave) of the user between the time of solving the high difficulty level questions and the time of solving the low difficulty level questions. The task difference Δtv [s] is a vector volume obtained by subtracting dispersion of reaction times of the user at the time of solving low difficulty level questions from dispersion of reaction times of the user at the time of solving the high difficulty level questions. The task difference ΔP is a vector volume obtained by subtracting a peak value of the power of the slow brain wave (α-wave) of the user at the time of solving the low difficulty level questions from a peak value of the power of the slow brain wave (α-wave) of the user at the time of solving the high difficulty level questions. It is to be noted that the kind of the dispersion of the reaction times is not limited to 75% percentile-25% percentile, and may be, for example, standard deviation.

FIG. 24 illustrates an example of a relationship between the task difference Δtv [s] and a task difference ΔR [%]. The task difference Δtv [s] is the task difference in the dispersion (75% percentile-25% percentile) of the reaction times of the user between the time of solving the high difficulty level questions and the time of solving the low difficulty level questions. The task difference ΔR [%] is a task difference in an accuracy rate for questions between the time of solving the high difficulty level questions and the time of solving the low difficulty level questions. The task difference ΔR is a vector volume obtained by subtracting the accuracy rate at the time of solving the low difficulty level questions from the accuracy rate at the time of solving the high difficulty level questions. It is to be noted that the kind of the dispersion of the reaction times is not limited to 75% percentile-25% percentile, and may be, for example, standard deviation.

In FIGS. 23 and 24, data for respective users are plotted, and features of the entirety of users are represented by a regression formula (regression line). In FIG. 23, the regression formula is represented by ΔP=a1×Δtv+b1, and in FIG. 24, the regression formula is represented by ΔR=a2×Δtv+b2.

A small task difference Δtv in the dispersion of the reaction times means that the difference in the dispersion of the reaction times between the time of solving the high difficulty level questions and the time of solving the low difficulty level questions is small. It can be said that a user who has obtained such a result has a tendency in which, as the difficulty level of the questions becomes high, the task difference in dispersion of time periods for solving questions becomes smaller as compared with other users. Meanwhile, a large task difference Δtv in the dispersion of the reaction times means that the difference in the dispersion of the reaction times between the time of solving the high difficulty level questions and the time of solving the low difficulty level questions is large. It can be said that a user who has obtained such a result has a tendency in which, as the difficulty level of the questions becomes high, the task difference in dispersion of time periods for solving questions becomes larger as compared with other users.

It is appreciated from FIG. 23 that, when the task difference Δtv in the dispersion of the reaction times is small, the task difference ΔP in the peak value of the power of the slow brain wave (α-wave) becomes large, and that, when the task difference Δtv in the dispersion of the reaction times is large, the task difference ΔP in the peak value of the power of the slow brain wave (α-wave) becomes small. It is appreciated from the above that a person who is able to answer even difficult questions within the same degree of reaction time as that for simple questions has a tendency in which the task difference ΔP in the peak value of the power of the slow brain wave (α-wave) becomes large. Conversely, it is appreciated that a person who has large dispersion of reaction times for difficult questions has a tendency in which the task difference ΔP in the peak value of the power of the slow brain wave (α-wave) does not vary so much regardless of the difficulty level of the questions.

It is appreciated from FIG. 24 that, when the task difference Δtv in the dispersion of the reaction times is large, the task difference ΔR in the accuracy rate for questions becomes small, and that, when the task difference Δtv in the dispersion of the reaction times is small, the task difference ΔR in the accuracy rate for questions becomes large. It is appreciated from the above that a person who has large dispersion of the reaction times for difficult questions has a tendency in which the task difference ΔR in the accuracy rate becomes small (i.e., the accuracy rate for difficult questions is lowered). Conversely, it is appreciated that a person who has small dispersion of the reaction times even for difficult questions has a tendency in which the task difference ΔR in the accuracy rate becomes large (i.e., is able to answer accurately even for difficult questions to the same degree as for simple questions).

It can be inferred from the above that, when the task difference Δtv in the dispersion of the reaction times is large, a cognitive capacity (cognitive resource) of the user is lower than a predetermined standard. In addition, it can be inferred that, when the task difference Δtv in the dispersion of the reaction times is small, the cognitive capacity of the user is higher than the predetermined standard. In a case where the cognitive capacity of the user is lower than the predetermined standard, the difficulty level of the questions may possibly be too high for the user. Meanwhile, in a case where the cognitive capacity of the user is higher than the predetermined standard, the difficulty level of questions may possibly be too low for the user.

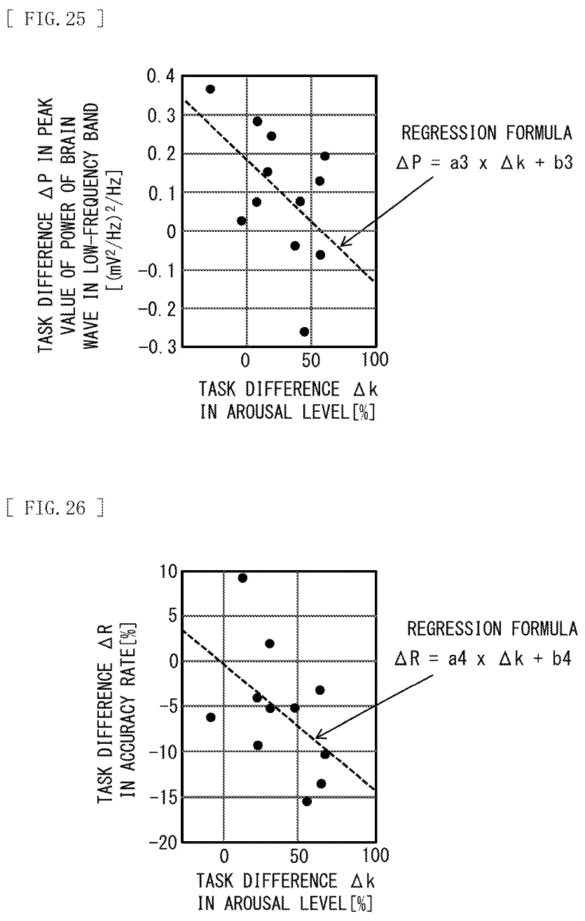

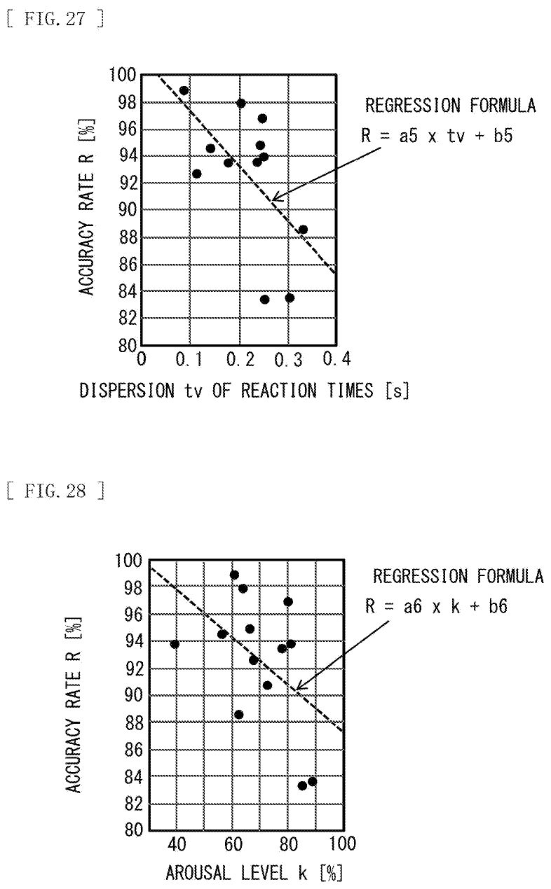

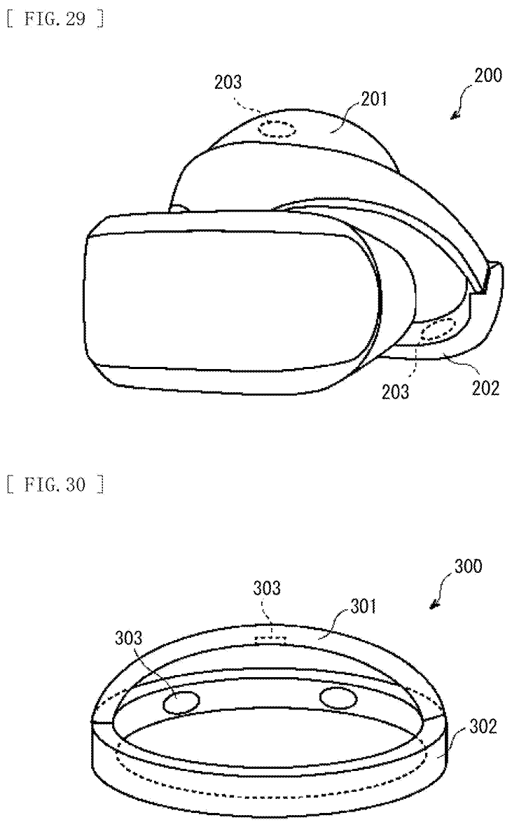

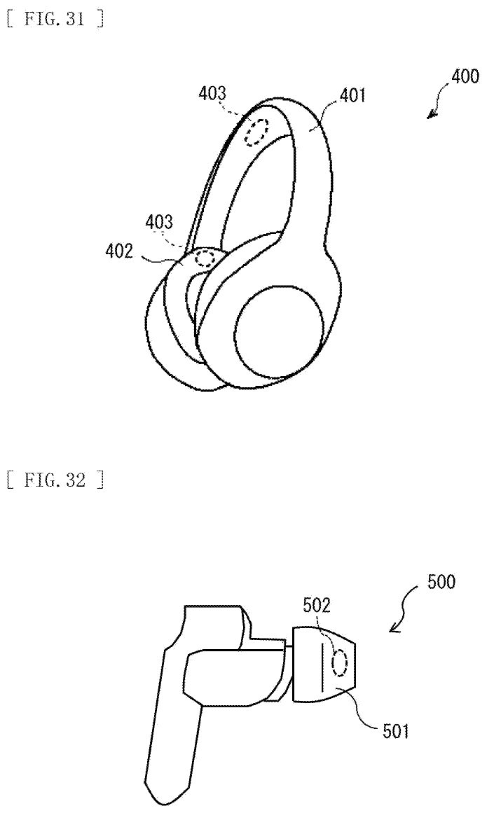

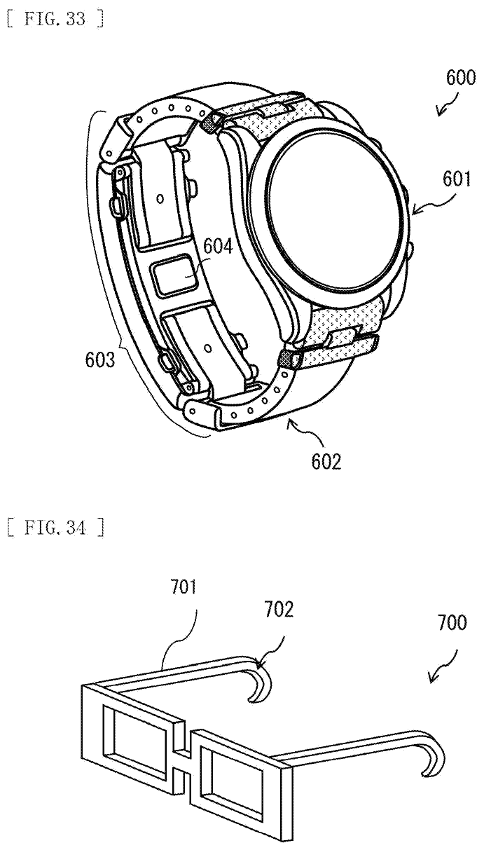

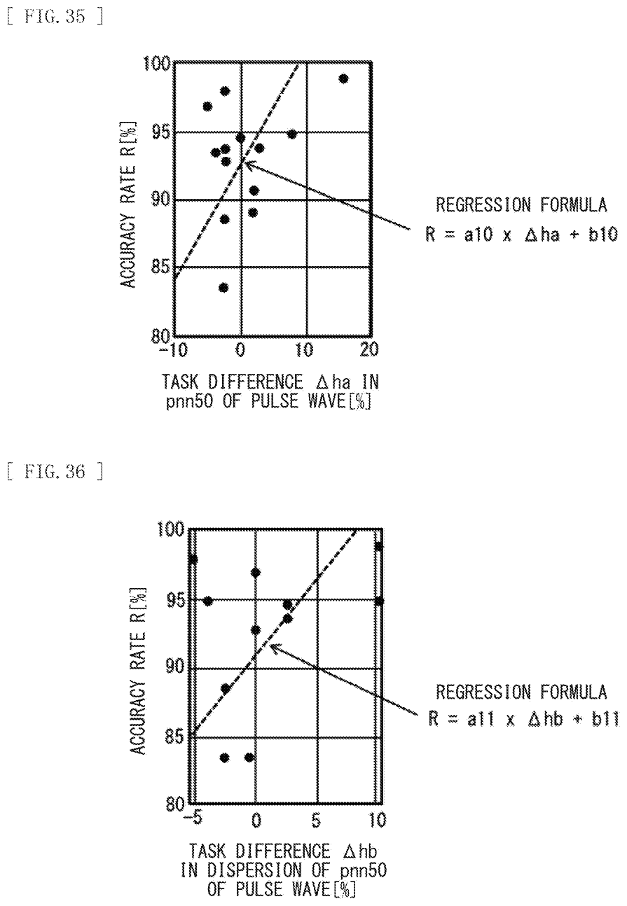

FIG. 25 illustrates an example of a relationship between a task difference Δk [%] and the task difference ΔP [mV²/Hz)²/Hz]. The task difference Δk [%] is a task difference in an arousal level of the user between the time of solving the high difficulty level questions and the time of solving the low difficulty level questions. The task difference ΔP [mV²/Hz)²/Hz] is the task difference in the peak value of the power of the slow brain wave (α-wave) of the user between the time of solving the high difficulty level questions and the time of solving the low difficulty level questions. FIG. 26 illustrates an example of a relationship between the task difference Δk [%] and the task difference ΔR [%]. The task difference Δk [%] is the task difference in the arousal level of the user between the time of solving the high difficulty level questions and the time of solving the low difficulty level questions. The task difference ΔR [%] is the task difference in the accuracy rate for questions between the time of solving the high difficulty level questions and the time of solving the low difficulty level questions. The task difference Δk [%] is a vector volume obtained by subtracting the arousal level of the user at the time of solving the low difficulty level questions from the arousal level of the user at the time of solving the high difficulty level questions. The arousal level is obtained, for example, by using the above-described estimation model that estimates the arousal level with use of the brain wave.

In FIGS. 25 and 26, data for respective users are plotted, and features of the entirety of users are represented by a regression formula (regression line). In FIG. 25, the regression formula is represented by ΔP=a3×Δk+b3, and in FIG. 26, the regression formula is represented by ΔR=a4×Δk+b4.

It is appreciated from FIGS. 23 to 26 that the task difference Δtv in the dispersion of the reaction times and task difference Δk in the arousal level have a correspondence relationship. Accordingly, it is appreciated that it is possible to estimate the task difference Δk in the arousal level by measuring the task difference Δtv in the dispersion of the reaction times.

FIG. 27 illustrates an example of a relationship between the dispersion (75% percentile-25% percentile) tv [s] of the reaction times of the user at the time of solving the high difficulty level questions and the accuracy rate R [%] for questions at the time of solving the high difficulty level questions. In FIG. 27, data for respective users are plotted, and features of the entirety of users are represented by a regression formula (regression line). In FIG. 27, the regression formula is represented by R=a5×tv+b5.

FIG. 28 illustrates an example of a relationship between the arousal level k [%] of the user at the time of solving the high difficulty level questions and the accuracy rate R [%] for questions at the time of solving the high difficulty level questions. In FIG. 28, data for respective users are plotted, and features of the entirety of users are represented by a regression formula (regression line). In FIG. 28, the regression formula is represented by R=a6×k+b6.

It is appreciated from FIGS. 27 and 28 that the dispersion tv of the reaction times and the arousal level k have a correspondence relationship. Accordingly, it is appreciated that it is possible to estimate the arousal level k by measuring the dispersion tv of the reaction times.

<2. About Comfort/Discomfort>

Comfort/discomfort of a person is closely related to concentration of the person. A person in concentration is interested and concerned in an object on which the person is concentrated. Accordingly, it is possible to estimate an objective interest/concern degree (emotion) of the person by knowing comfort/discomfort of the person. It is possible to derive comfort/discomfort of the person on the basis of biological information or motion information acquired from the person who is in conversation with a communication partner or the communication partner (hereinafter referred to as a “target living body”).

Examples of biological information from which comfort/discomfort of the target living body is derivable include information about a brain wave and sweating. In addition, examples of motion information from which the comfort/discomfort of the target living body is derivable include facial expression.

(Brain Wave)

It is known that it is possible to estimate comfort/discomfort of a person from a difference in an α-wave included in a brain wave between a left forehead and a right forehead. Accordingly, for example, an α-wave included in a brain wave obtained in the left forehead (hereinafter referred to as a “left-side α-wave”) and an α-wave included in a brain wave obtained in the right forehead (hereinafter referred to as a “right-side α-wave”) are compared with each other. In this case, it is possible to estimate that, when the left-side α-wave is lower than the right-side α-wave, the target living body feels comfort, and that, when the left-side α-wave is higher than the right-side α-wave, the target living body feels discomfort.

In addition, in estimating the comfort/discomfort of the target living body with use of the brain wave, it is possible to use an estimation model such as machine learning in place of deriving a difference in the α-wave included in the brain wave between the left forehead and the right forehead. This estimation model is, for example, a model trained using, as teaching data, an α-wave or a β-wave included in a brain wave when the target living body apparently feels comfort. For example, in a case where an α-wave or a β-wave included in a brain wave is inputted, this estimation model estimates the comfort/discomfort of the target living body on the basis of the inputted α-wave or the inputted β-wave. This estimation model includes, for example, a neural network. This learning model may include, for example, a deep neural network such as a convolutional neural network (CNN).

(Sweating)

Emotional sweating is sweating released from eccrine sweat glands when sympathetic nerves are strained due to a mental/psychological issue such as stress, tension, or anxiety. For example, a sweat sensor probe is attached to a palm or a sole, and sweating (emotional sweating) on the palm or the sole induced by various load stimuli is measured, which makes it possible to obtain a sympathetic sweat response (SSwR) as a signal voltage. When, in this signal voltage, a numeric value of a predetermined high-frequency component or a predetermined low-frequency component obtained from a left hand is higher than a numeric value of the predetermined high-frequency component or the predetermined low-frequency component obtained from a right hand, it is possible to estimate that the target living body feels comfort. In addition, when, in the signal voltage described above, the numeric value of the predetermined high-frequency component or the predetermined low-frequency component obtained from the left hand is lower than the numeric value of the predetermined high-frequency component or the predetermined low-frequency component obtained from the right hand, it is possible to estimate that the target living body feels discomfort. In addition, when, in this signal voltage, an amplitude value obtained from the left hand is higher than an amplitude value obtained from the right hand, it is possible to estimate that the target living body feels comfort. In addition, when, in the signal voltage described above, the amplitude value obtained from the left hand is lower than the amplitude value obtained from the right hand, it is possible to estimate that the target living body feels discomfort.

In addition, it is possible to estimate the arousal level of the target living body with use of, for example, an estimation model that estimates the arousal level of the target living body on the basis of the predetermined high-frequency component or the low-frequency component included in this signal voltage. This estimation model is, for example, a model trained using, as teaching data, the predetermined high-frequency component or the predetermined low-frequency component included in a signal voltage when the arousal level is apparently high. For example, in a case where the predetermined high-frequency component or the predetermined low-frequency component is inputted, this estimation model estimates the arousal level of the target living body on the basis of the inputted predetermined high-frequency component or the inputted predetermined low-frequency component. This estimation model includes, for example, a neural network. This learning model may include, for example, a deep neural network such as a convolutional neural network (CNN).

(Facial Expression)

It is known that eyebrows are knitted when feeling discomfort, and that zygomaticus major muscles hardly change when feeling comfort. Thus, it is possible to estimate comfort/discomfort in accordance with facial expression. Accordingly, for example, a face is photographed by a camera to estimate facial expression on the basis of thus-acquired moving image data, and it is possible to estimate the comfort/discomfort of the target living body in accordance with the facial expression obtained by the estimation.

In addition, it is possible to estimate the comfort/discomfort of the target living body with use of, for example, an estimation model that estimates the comfort/discomfort of the target living body on the basis of moving image data acquired by photographing facial expression. This estimation model is, for example, a model trained using, as teaching data, moving image data acquired by photographing facial expression when the arousal level is apparently high. For example, in a case where moving image data acquired by photographing facial expression is inputted, this estimation model estimates the comfort/discomfort of the target living body on the basis of the inputted moving image data. This estimation model includes, for example, a neural network. This learning model may include, for example, a deep neural network such as a convolutional neural network (CNN).

For example, the following literature describes a frequency component of a brain wave.
Wang, Xiao-Wei, Dan Nie, and Bao-Liang Lu. “EEG-based emotion recognition using frequency domain features and support vector machines.” International conference on neural information processing. Springer, Berlin, Heidelberg, 2011.

For example, the following literature describes an estimation model using a brain wave.
Japanese Patent Application No. 2020-203058

For example, the following literatures describe sweating.
Jing Zhai, A. B. Barreto, C. Chin and Chao Li, “Realization of stress detection using psychophysiological signals for improvement of human-computer interactions,” Proceedings. IEEE SoutheastCon, 2005., Ft. Lauderdale, FL, USA, 2005, pp. 415-420, doi: 10.1109/SECON.2005.1423280.
Boucsein, Wolfram. Electrodermal activity. Springer Science & Business Media, 2012. For example, the following literature describes the heart rate.
Veltman, J. A., and A. W. K. Gaillard. “Physiological indices of workload in a simulated flight task.” Biological psychology 42.3 (1996): 323-342.

For example, the following literature describes heart rate variability intervals.
Appelhans, Bradley M., and Linda J. Luecken. “Heart rate variability as an index of regulated emotional responding.” Review of general psychology 10.3 (2006): 229-240.

For example, the following literature describes a cortisol content in saliva.
Lam, Suman, et al. “Emotion regulation and cortisol reactivity to a social-evaluative speech task.” Psychoneuroendocrinology 34.9 (2009): 1355-1362.

For example, the following literature describes facial expression.
Lyons, Michael J., Julien Budynek, and Shigeru Δkamatsu. “Automatic classification of single facial images.” IEEE transactions on pattern analysis and machine intelligence 21.12 (1999): 1357-1362.

For example, the following literature describes facial muscles.
Ekman, Paul. “Facial action coding system.” (1977).

For example, the following literature describes a blinking frequency
Chen, Siyuan, and Julien Epps. “Automatic classification of eye activity for cognitive load measurement with emotion interference.” Computer methods and programs in biomedicine 110.2 (2013): 111-124.

For example, the following literature describes respiratory volume/respiration rate.
Zhang Q., Chen X., Zhan Q., Yang T., Xia S. Respiration-based emotion recognition with deep learning. Comput. Ind. 2017; 92-93:84-90. doi: 10.1016/j.compind.2017.04.005.

For example, the following literature describes a skin surface temperature.
Nakanishi R., Imai-Matsumura K. Facial skin temperature decreases in infants with joyful expression. Infant Behay. Dev. 2008; 31:137-144. doi: 10.1016/j.infbeh.2007.09.001.

For example, the following literature describes multimodal.
Choi J.-S., Bang J., Heo H., Park K. Evaluation of Fear Using Nonintrusive Measurement of Multimodal Sensors. Sensors. 2015; 15:17507-17533. doi: 10.3390/s150717507.

In the following, description is given of embodiments of an information processing system using an derivation algorithm of the arousal level or the comfort/discomfort described above.

### 2. First Embodiment

[Configuration]

Description is given of a biological information processing system 100 according to a first embodiment of the present disclosure. FIG. 1 illustrates a schematic configuration example of the biological information processing system 100. The biological information processing system 100 is an objective evaluation system that evaluates the target living body on the basis of at least one of biological information or action information acquired from the target living body. In the present embodiment, the target living body is a human. It is to be noted that, in the biological information processing system 100, the target living body is not limited to the human.

The biological information processing system 100 includes a biological sensor 10 that detects biological information about a person to be evaluated, and an electronic apparatus 20 that processes a detection signal outputted from the biological sensor 10. The biological sensor 10 and the electronic apparatus 20 are coupled to each other to enable data transmission/reception via a network 30. The network 30 is a wireless or wired communication means, and examples thereof include the Internet, a WAN (Wide Area Network), a LAN (Local Area Network), a public communication network, a dedicated line, and the like.

The biological sensor 10 may be, for example, a sensor of a type that comes in contact with the person to be evaluated or a sensor that is noncontact with the person to be evaluated. The biological sensor 10 is, for example, a sensor that acquires information (biological information) about at least one of a brain wave, sweating, a pulse wave, an electrocardiogram, a blood flow, a skin temperature, potential of facial muscles, eye potential, or a specific component contained in saliva. The biological sensor 10 may be, for example, a sensor that acquires information (action information) about at least one of facial expression, voice, or a reaction time. The biological sensor 10 may be, for example, a sensor that acquires at least one piece of information of the biological information or the action information. The biological sensor 10 outputs the acquired information (at least one piece of information of the biological information or the action information) to the electronic apparatus 20.

The electronic apparatus 20 includes a sensor input receiver 21, a user input receiver 22, a signal processor 23, a storage section 24, an image data generator 25, and an image display section 26. The signal processor 23 corresponds to a specific example of each of a “derivation section”, a “classification section”, a “receiver”, and a “selector” of the present disclosure. The storage section 24 corresponds to a specific example of a “storage section” of the present disclosure. The image data generator 25 corresponds to a specific example of an “image data generator” of the present disclosure.

The sensor input receiver 21 receives an input from the biological sensor 10, and outputs the input to the signal processor 23. The input from the biological sensor 10 includes at least one of the biological information or the action information. The sensor input receiver 21 includes, for example, an interface that is able to communicate with the biological sensor 10. The user input receiver 22 receives an input from a user, and outputs the input to the signal processor 23. Examples of the input from the user include attribute information (e.g., a name or the like) about the person to be evaluated, and an evaluation start instruction. The user input receiver 22 includes, for example, an input interface such as a keyboard, a mouse, or a touch panel.

The storage section 24 is, for example, a volatile memory such as a DRAM (Dynamic Random Access Memory), or a non-volatile memory such as an EEPROM (Electrically Erasable Programmable Read-Only Memory) or a flash memory. A biological information processing program 24a for evaluating the person to be evaluated, and task data and an classification index 24c that are used in the biological information processing program 24a are stored in the storage section 24. The classification index 24c corresponds to a specific example of a “predetermined classification index” of the present disclosure. Furthermore, an identifier 24d, an arousal level 24e, a feature amount 24f, a classification result 24g, and an evaluation result 24h that are obtained by processing by the biological information processing program 24a are stored in the storage section 24. Details of the processing in the biological information processing program 24a are described in detail later.

The task data 24b includes, for example, a plurality of pieces of question data. The plurality of pieces of question data is tasks to be assigned to the person to be evaluated while acquiring the biological information about the person to be evaluated, and corresponds to a specific example of a “specific task” of the present disclosure. The task data 24b may be omitted as necessary. In this case, as tasks to be assigned to the person to be evaluated while acquiring the biological information about the person to be evaluated, for example, a an apparatus (e.g., an electronic apparatus for a test, or a game machine) provided separately from the electronic apparatus 20, a paper medium (e.g., a test paper), or the like may be prepared in advance. It is to be noted that in the following, tasks using the task data 24b are provided by the electronic apparatus 20.

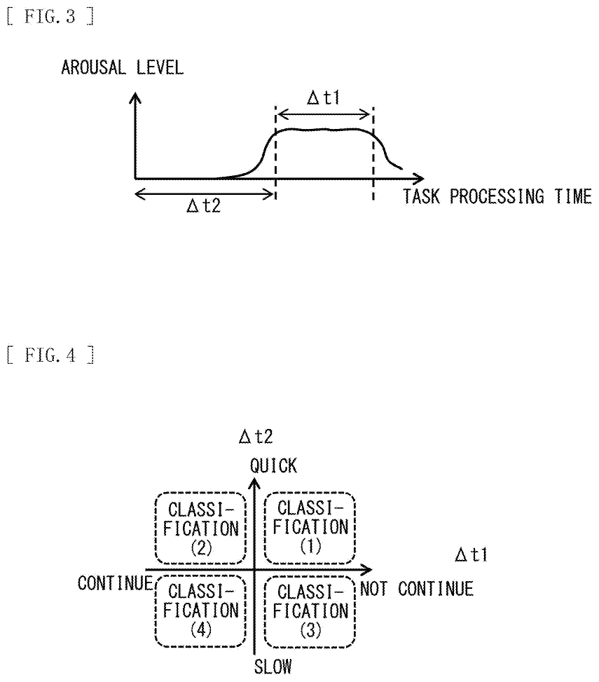

The classification index 24c includes one or a plurality of indices to be used for evaluation of the person to be evaluated, and includes, for example, duration of the arousal level and rise time of the arousal level. The duration of the arousal level indicates, for example, a period (duration Δt1) in which the arousal level is maintained in a high state, as illustrated in FIG. 3. The rise time of the arousal level indicates, for example, time (rise time Δt2) taken to change the arousal level from a low state to the high state, as illustrated in FIG. 3. The duration Δt1 is an index related to durability of concentration, and longer duration Δt1 indicates having an ability to maintain high concentration longer. The rise time Δt2 is an index related to quickness of on/off switching, and shorter rise time Δt2 indicates an ability to more quickly concentrate on a work.

The identifier 24d is numeric value data for identifying the person to be evaluated, and is, for example, an identification number assigned to each person to be evaluated. The identifier 24d is generated, for example, at a timing when the person to be evaluated inputs the attribute information about the person to be evaluated. The arousal level 24e is numerical data about the arousal level derived on the basis of an input (detection signal) from the biological sensor 10. The arousal level 24e is, for example, numerical data about the arousal level that changes over time, as illustrated in FIG. 3. The feature amount 24f is numerical data about one or a plurality of indices included in the classification index 24c.

The feature amount 24f includes, for example, the duration Δt1 and the rise time Δt2 derived from the arousal level 24e. The classification result 24g indicates one classification of a plurality of classifications classified in accordance with magnitude of the feature amount 24f (e.g., the lengths of the duration Δt1 and the rise time Δt2). Examples of the plurality of classifications include classifications as illustrated in FIG. 4.
Classification (1): A classification in which both the duration Δt1 and the rise time Δt2 are large
Classification (2): A classification in which the duration Δt1 is small and the rise time Δt2 is large
Classification (3): A classification in which the duration Δt1 is large and the rise time Δt2 is small
Classification (4): A classification in which both the duration Δt1 and the rise time Δt2 are small

The evaluation result 24h is a suitability/unsuitability evaluation result in recruitment activity or selection of a person for team building in an organization. The evaluation result 24h is, for example, a result obtained by evaluation on the basis of the classification result 24g. For example, in a case where the feature amount 24f corresponds to the classification (1), the evaluation result 24h is “suitability”. In addition, for example, in a case where the feature amount 24f corresponds to the classification (4), the evaluation result 24h is “unsuitability”.

The signal processor 23 includes, for example, a processor. The signal processor 23 executes the biological information processing program 24a stored in the storage section 24. A function of the signal processor 23 is implemented, for example, by executing the biological information processing program 24a by the signal processor 23. The signal processor 23 executes a series of processing necessary for evaluation of the person to be evaluated. The signal processor 23 reads, for example, a predetermined plurality of pieces of question data from the task data 24b, and sequentially outputs the read plurality of pieces of question data to the image data generator 25. The image data generator 25 generates image data including the pieces of question data inputted from the signal processor 23, and outputs the image data to the image display section 26. The image display section 26 displays an image on the basis of the image data inputted from the image data generator 25. The person to be evaluated solves questions while watching the image displayed on the image display section 26.

The person to be evaluated inputs, for example, answers corresponding to the pieces of question data to the user input receiver 22. For example, upon acquiring an answer corresponding to a question displayed on the image display section 26 from the user input receiver 22, the signal processor 23 outputs the next piece of question data to the image data generator 25. It is to be noted that the person to be evaluated may write an answer corresponding to a piece of question data on a paper sheet and input a notification that answering is completed to the user input receiver 22. In this case, for example, upon receiving the notification that answering is completed from the user input receiver 22, the signal processor 23 outputs the next piece of question data to the image data generator 25. The person to be evaluated may write an answer corresponding to a piece of question data on a paper sheet and input nothing to the user input receiver 22. In this case, the signal processor 23 outputs the next piece of question data to the image data generator 25 at regular intervals, for example.

The signal processor 23 derives the arousal level 24e of the person to be evaluated, on the basis of at least one piece of information of the biological information or the action information acquired from the person to be evaluated while the person to be evaluated is performing a task (specific task) of solving a plurality of questions. In this case, the signal processor 23 derives the arousal level 24e of the person to be evaluated with use of one method of various methods described above. The signal processor 23 derives, for example, time series data as the arousal level 24e. For example, the signal processor 23 further stores, in the storage section 24, the derived time series data in association with the identifier 24d of the person to be evaluated.

The signal processor 23 derives the feature amount 24f corresponding to the classification index 24c on the basis of the arousal level 24e. The signal processor 23 derives, for example, the duration Δt1 and the rise time Δt2 from the arousal level 24e. The signal processor 23 selects one classification from among the plurality of classifications (1) to (4) in accordance with, for example, the magnitude of the derived feature amount 24f (e.g., the lengths of the duration Δt1 and the rise time Δt2). The signal processor 23 evaluates the person to be evaluated, on the basis of, for example, the selected classification (classification result 24g). The signal processor 23 stores, for example, the evaluation result 24h of the person to be evaluated in the storage section 24.

Evaluation criteria for the person to be evaluated are stored in the storage section 24. The evaluation criteria for the person to be evaluated include, for example, recruitment criteria or criteria for team building in the organization. In general, the recruitment criteria often include attribute information such as the age, gender, and educational background of a person. In addition, the criteria for team building in the organization often include human intuition, experiences, and subjectivity. However, in the present embodiment, the recruitment criteria and the criteria for team building in the organization are based on the classification result 24g. For example, the recruitment criteria are that the classification result 24g corresponds to the classification (1). The criteria for team building in the organization also considers a relationship with other members; therefore, the criteria for team building in the organization may be different from the recruitment criteria. For example, the criteria for team building in the organization include three persons having the classification result 24g corresponding to the classification (1), one person having the classification result 24g corresponding to the classification (2), one person having the classification result 24g corresponding to the classification (3), and one person having the classification result 24g corresponding to the classification (4).

The biological information processing system 100 may evaluate an applicant (person to be evaluated) for recruitment. In this case, for evaluation of the person to be evaluated, the signal processor 23 assigns the identifier 24d to the person to be evaluated, and stores the assigned identifier 24d in the storage section 24. Upon obtaining the arousal level 24e, the signal processor 23 stores, in the storage section 24, the classification result 24g derived from the obtained arousal level 24e in association with the assigned identifier 24d. In a case where the classification result 24g stored in the storage section 24 meets the recruitment criteria, the signal processor 23 stores, in the storage section 24, the identifier 24d of a person who meets the recruitment criteria as the evaluation result 24h.

The biological information processing system 100 may sequentially evaluate a plurality of persons to be evaluated to select persons suitable to constitute a specific group (e.g., a team in an organization). In this case, every time the signal processor 23 evaluates the person to be evaluated, the signal processor 23 assigns the identifier 24d to the person to be evaluated, and stores the assigned identifier 24d in the storage section 24. Every time the signal processor 23 obtains the arousal level 24e, the signal processor 23 stores, in the storage section 24, the classification result 24g derived from the obtained arousal level 24e in association with the assigned identifier 24d. The signal processor 23 selects a plurality of identifiers 24d suitable to constitute the specific group (e.g., the team in the organization) on the basis of the respective classification results 24g of the plurality of persons to be evaluated as evaluation targets stored in the storage section 24. The signal processor 23 extracts classification results 24g that meet criteria for constituting the specific group (e.g., the team in the organization) from among a plurality of classification results 24g, corresponding to the plurality of persons to be evaluated, stored in the storage section 24. The signal processor 23 stores, in the storage section 24, a plurality of identifiers 24d corresponding to a plurality of extracted classification results 24g as the evaluation results 24h.

The image data generator 25 generates image data in which the classification index 24c and the feature amount 24f derived for evaluation are associated with each other. The image data generator 25 generates image data in which the classification index 24c and time series data of the arousal level 24e are associated with each other. The image data generator 25 outputs the generated image data to the image display section 26.

The image display section 26 displays an image based on the image data inputted from the image data generator 25. In this case, the image display section 26 displays, for example, an image as illustrated in FIG. 4 on the display screen 26A. On the display screen 26A, for example, as illustrated in FIG. 4, the classification indices 24c are displayed in the form of a two-dimensional graph, and the feature amount 24f is displayed as a plot in any quadrant of the two-dimensional graph. On the display screen 26A, for example, as illustrated in FIG. 4, the time series data of the arousal level 24e is displayed as a waveform.

In a case where the plurality of persons to be evaluated is sequentially evaluated to select persons suitable to constitute the specific group (e.g., the team in then organization), the image data generator 25 generates image data in which the classification index 24c and a plurality of feature amounts 24f derived for evaluation of the plurality of persons to be evaluated are associated with each other. The image data generator 25 generates image data in which the classification index 24c and pieces of time series data of the arousal levels 24e of the plurality of persons to be evaluated are associated with each other. The image data generator 25 outputs the generated image data to the image display section 26.

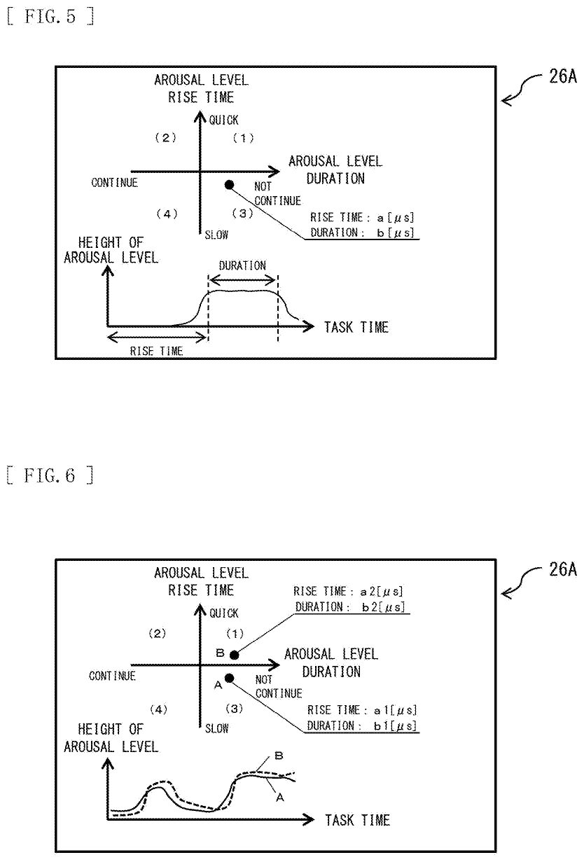

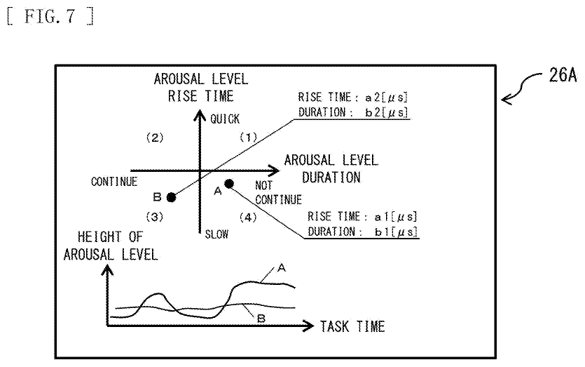

The image display section 26 displays an image based on the image data inputted from the image data generator 25. In this case, the image display section 26 displays, for example, an image as illustrated in FIGS. 5 and 6 on the display screen 26A. On the display screen 26A, for example, as illustrated in FIGS. 5 and 6, the classification indices 24c are displayed in the form of a two-dimensional graph, and the plurality of feature amounts 24f is displayed as plots in one or a plurality of quadrants of the two-dimensional graph. For example, as illustrated in FIGS. 5 and 6, pieces of time series data of a plurality of arousal levels 24e are displayed on the display screen 26A to be aligned in time and superimposed on each other.

It is to be noted that FIG. 5 exemplifies waveforms when the pieces of time series data of the plurality of arousal levels 24e are nearly synchronized with each other. FIG. 6 exemplifies waveforms when the pieces of time series data of the plurality of arousal levels 24e are not synchronized with each other at all. When the pieces of time series data of the plurality of arousal levels 24e are nearly synchronized with each other as illustrated in FIG. 6, a plurality of persons to be evaluated of which the arousal levels 24e have been calculated may be classified into a common classification index 24c. Meanwhile, as illustrated in FIG. 7, when the pieces of time series data of the plurality of arousal levels 24e are not synchronized with each other at all, a plurality of persons to be evaluated of which the arousal levels 24e have been calculated may be classified into classification indices 24c different from each other. Accordingly, the user is able to evaluate the plurality of persons to be evaluated of which the arousal levels 24e have been calculated from synchronization of the pieces of time series data of the plurality of arousal levels 24e displayed on the image display section 26. When an image (the pieces of times series data of the plurality of arousal levels 24e) as illustrated in FIGS. 6 and 7 is displayed on the image display section 26, the signal processor 23 receives selection of a plurality of arousal levels 24e from among the plurality of arousal levels 24e or selection of a plurality of identifiers 24d from among the plurality of identifiers 24d via the user input receiver 22. The signal processor 23 selects a plurality of identifiers 24d suitable to constitute the specific group (e.g., the team in the organization) on the basis of received contents (selection result). The signal processor 23 stores, in the storage section 24, the selected plurality of identifiers 24d as the evaluation results 24h. Thus, the user is able to evaluate a plurality of persons to be evaluated of which the arousal levels 24e have been calculated, from synchronization of the pieces of time series data of the plurality of arousal levels 24e displayed on the image display section 26.

[Operation]

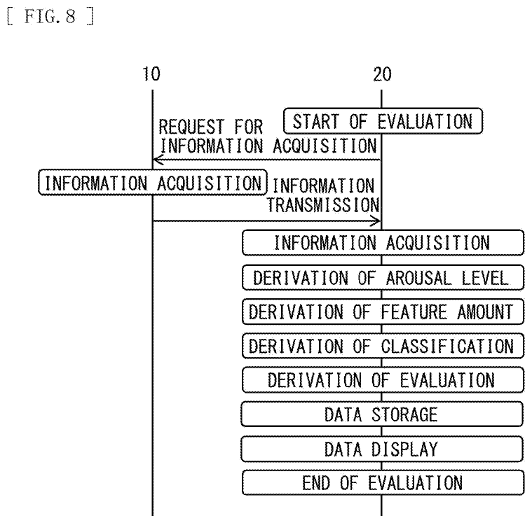

Next, description is given of an operation of the biological information processing system 100. FIG. 8 illustrates an example of an evaluation procedure in the biological information processing system 100.

First, the electronic apparatus 20 (signal processor 23) loads the biological information processing program 24a from the storage section 24, and starts execution of a series of procedures for evaluation described in the biological information processing program 24a. The signal processor 23 reads a predetermined plurality of pieces of question data from the task data 24b, and sequentially outputs the read plurality of pieces of question data to the image data generator 25. The image data generator 25 generates image data including the pieces of question data inputted from the signal processor 23, and outputs the image data to the image display section 26. The image display section 26 displays an image on the basis of the image data inputted from the image data generator 25. In this case, the person to be evaluated solves questions while watching the image displayed on the image display section 26.

The signal processor 23 outputs a request for information acquisition to the biological sensor 10. The request for information acquisition is a series of control signals for causing the biological sensor 10 to acquire at least one piece of information of biological information or action information about the person to be evaluated while the person to be evaluated is performing a task (specific task) of solving a plurality of questions. The biological sensor 10 acquires at least one piece of information of the biological information or the action information in response to the input of the request for information acquisition, and outputs the at least one piece of information to the electronic apparatus 20.

Upon acquiring the information (at least one piece of information of the biological information or the action information) from the biological sensor 10, the electronic apparatus 20 (signal processor 23) derives the arousal level 24e on the basis of the acquired information. The signal processor 23 derives the feature amount 24f corresponding to the classification index 24c on the basis of the derived arousal level 24e. The signal processor 23 selects one classification from among the plurality of classifications (1) to (4) in accordance with the magnitude of the derived feature amount 24f (e.g., the lengths of the duration Δt1 and the rise time Δt2). The signal processor 23 evaluates the person to be evaluated on the basis of the selected classification (classification result 24g). The signal processor 23 stores, in the storage section 24, for example, the evaluation result 24h of the person to be evaluated.

The image data generator 25 generates image data in which the classification index 24c and the feature amount 24f derived for evaluation are associated with each other. The image data generator 25 generates image data in which the classification index 24c and the time series data of the arousal level 24e are associated with each other. The image data generator 25 outputs the generated image data to the image display section 26. The image display section 26 displays an image based on the image data inputted from the image data generator 25. The image display section 26 displays, for example, an image as illustrated in FIGS. 5 to 7 on the display screen 26A.

### Effects

Next, description is given of effects of the biological information processing system 100.

In the present embodiment, the arousal level 24e is classified on the basis of the predetermined classification index 24c. This makes it possible to classify the person to be evaluated with use of the arousal level 24e that is objective data. As a result, for example, in a case of recruitment, it is possible to determine, from the arousal level 24e of the person to be evaluated, whether or not the person to be evaluated is a desired talent. In addition, for example, in a case of determining project members, it is possible to determine, from the arousal levels 24e of a large number of persons to be evaluated, whether or not the persons to be evaluated are members suitable to constitute a specific group. It is therefore possible to reduce mismatches.

In the present embodiment, the person to be evaluated is evaluated on the basis of the classification result 24g. Herein, the classification result 24g is derived from the arousal level 24e that is objective data. Accordingly, for example, in a case of recruitment, it is possible to determine, from the objective data, whether the person to be evaluated is a desired talent. It is therefore possible to reduce mismatches.

In the present embodiment, every time the arousal level 24e is obtained, the classification result 24g derived from the arousal level 24e is stored, in the storage section 24, in association with the identifier 24d of the person to be evaluated. Furthermore, a plurality of identifiers 24d suitable to constitute the specific group is selected on the basis of a plurality of classification results 24g stored in the storage section 24. Accordingly, for example, in a case of determining project members, it is possible to determine, from the objective data, whether the persons to be evaluated are members suitable to constitute the specific group. It is therefore possible to reduce mismatches.

In the present embodiment, the feature amount 24f corresponding to the classification index 24c is derived on the basis of the arousal level 24e, and the derived feature amount 24f is stored, in the storage section 24, in association with the identifier 24d of the person to be evaluated. This makes it possible to classify the person to be evaluated with use of the feature amount 24f that is objective data. As a result, for example, in a case of recruitment, it is possible to determine, from the feature amount 24f of the person to be evaluated, whether or not the person to be evaluated is a desired talent. In addition, for example, in a case of determining project members, it is possible to determine, from the feature amounts 24f of a large number of persons to be evaluated, whether or not the persons to be evaluated are members suitable to constitute the specific group. It is therefore possible to reduce mismatches.

In the present embodiment, image data in which the classification index 24c and the feature amount 24f are associated with each other is generated. Accordingly, it is possible for the user to evaluate the person to be evaluated by watching an image displayed on the basis of the image data. As a result, for example, in a case of recruitment, it is possible to determine, from the feature amount 24f of the person to be evaluated, whether or not the person to be evaluated is a desired talent. In addition, for example, in a case of determining project members, it is possible to determine, from the feature amounts 24f of a large number of persons to be evaluated, whether or not the persons to be evaluated are members suitable to constitute the specific group. It is therefore possible to reduce mismatches.

In the present embodiment, time series data is derived as the arousal level 24e, and the derived time series data is stored, in the storage section 24, in association with the identifier 24d of the person to be evaluated. This makes it possible to classify the person to be evaluated with use of the arousal level 24e that is objective data. As a result, for example, in a case of recruitment, it is possible to determine, from the arousal level 24e of the person to be evaluated, whether or not the person to be evaluated is a desired talent. In addition, for example, in a case of determining project members, it is possible to determine, from the arousal levels 24e of a large number of persons to be evaluated, whether or not the persons to be evaluated are members suitable to constitute the specific group. It is therefore possible to reduce mismatches.

In the present embodiment, image data in which the classification index 24c and time series data of the arousal level 24e are associated with each other is generated. Accordingly, it is possible for the user to evaluate the person to be evaluated by watching an image displayed on the basis of the image data. As a result, for example, in a case of recruitment, it is possible to determine, from the time series data of the arousal level 24e of the person to be evaluated, whether or not the person to be evaluated is a desired talent. In addition, for example, in a case of determining project members, it is possible to determine, from synchronization of pieces of time series data of the arousal levels 24e of a large number of persons to be evaluated, whether or not the persons to be evaluated are members suitable to constitute the specific group. It is therefore possible to reduce mismatches.

In the present embodiment, the derived arousal level 24e is stored, in the storage section 24, in association with the identifier 24d of the person to be evaluated. This makes it possible to classify the person to be evaluated with use of the arousal level 24e that is objective data. As a result, for example, in a case of recruitment, it is possible to determine, from the arousal level 24e of the person to be evaluated, whether or not the person to be evaluated is a desired talent. In addition, for example, in a case of determining project members, it is possible to determine, from the arousal levels 24e of a large number of persons to be evaluated, whether or not the persons to be evaluated are members suitable to constitute the specific group. It is therefore possible to reduce mismatches.

In the present embodiment, every time the arousal level 24e is derived, the derived arousal level 24e is stored, in the storage section 24, in association with the identifier 24d of the person to be evaluated. Furthermore, image data in which the arousal levels 24e corresponding to a plurality of identifiers 24d are summarized in a mutually comparable manner is generated. Accordingly, it is possible for the user to evaluate the person to be evaluated by watching an image displayed on the basis of the image data. As a result, in a case of determining project members, it is possible to determine, from the arousal levels 24e of a large number of persons to be evaluated, whether or not the persons to be evaluated are members suitable to constitute the specific group. It is therefore possible to reduce mismatches.

In the present embodiment, selection of a plurality of arousal levels 24e from among the plurality of arousal levels 24e or selection of a plurality of identifiers 24d from among the plurality of identifiers 24d from the user is received. Then, a plurality of identifiers 24d suitable to constitute the specific group is selected on the basis of the received contents. Accordingly, for example, in a case of determining project members, it is possible to determine, from objective data, whether or not the persons to be evaluated are members suitable to constitute the specific group. It is therefore possible to reduce mismatches.

In the present embodiment, every time the arousal level 24e is derived, the derived arousal level 24e is stored, in the storage section 24, in association with the identifier 24d of the person to be evaluated. Furthermore, a plurality of identifiers 24d suitable to constitute the specific group is selected on the basis of a plurality of arousal level 24e stored in the storage section 24 and the predetermined classification index 24c. Accordingly, for example, in a case of determining project members, it is possible to determine, from objective data, whether or not the persons to be evaluated are members suitable to constitute the specific group. It is therefore possible to reduce mismatches.

In the present embodiment, time series data is derived as the arousal level 24e, and the derived time series data is stored, in the storage section 24, in association with the identifier 24d of the person to be evaluated. Then, image data in which pieces of time series data of the arousal levels 24e corresponding to a plurality of identifiers 24d are aligned in time and superimposed on each other is generated as image data. Accordingly, it is possible for the user to evaluate the person to be evaluated by watching an image displayed on the basis of the image data. As a result, in a case of determining project members, it is possible to determine, from the arousal levels 24e of a large number of persons to be evaluated, whether or not the persons to be evaluated are members suitable to constitute the specific group. It is therefore possible to reduce mismatches.

### 3. Second Embodiment

[Configuration]

Next, description is given of a biological information processing system 110 according to a second embodiment of the present disclosure. FIG. 9 illustrates a schematic configuration example of the biological information processing system 110. The biological information processing system 110 is an objective evaluation system that evaluates the target living body on the basis of at least one of biological information or action information acquired from the target living body. In the present embodiment, the target living body is a human. It is to be noted that, in the biological information processing system 110, the target living body is not limited to the human.

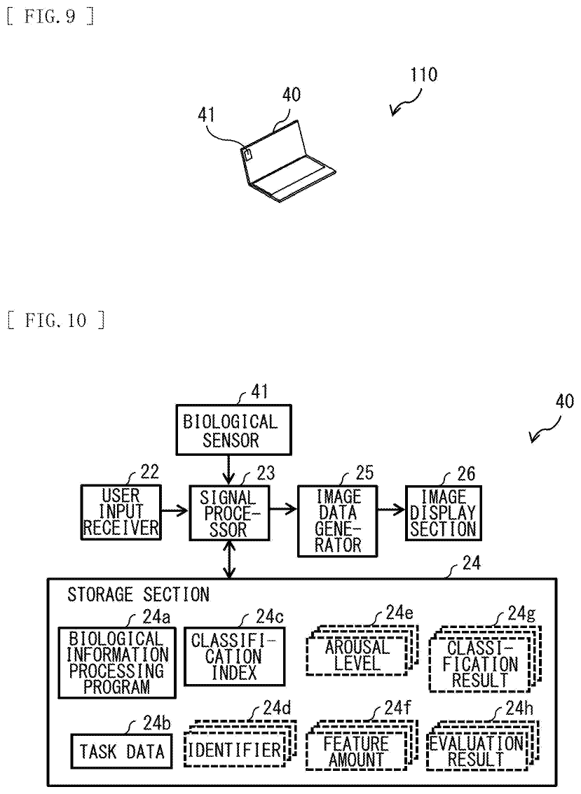

The biological information processing system 110 includes an electronic apparatus 40 including a biological sensor 41 that detects biological information about a person to be evaluated. The biological sensor 41 has a configuration similar to that of the biological sensor 10 according to the embodiment described above. The electronic apparatus 40 corresponds to, for example, the electronic apparatus 20 including the biological sensor 41 in place of the sensor input receiver 21, as illustrated in FIG. 10. The biological sensor 41 outputs acquired information (at least one piece of information of the biological information or the action information) to the signal processor 23.

[Operation]

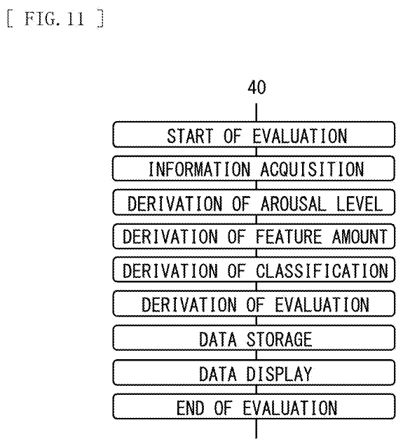

Next, description is given of an operation of the biological information processing system 110. FIG. 11 illustrates an example of an evaluation procedure in the biological information processing system 110.

First, the signal processor 23 loads the biological information processing program 24a from the storage section 24, and starts execution of a series of procedures for evaluation described in the biological information processing program 24a. The signal processor 23 reads, for example, a predetermined plurality of pieces of question data from the task data 24b, and sequentially outputs the read plurality of pieces of question data to the image data generator 25. The image data generator 25 generates image data including the pieces of question data inputted from the signal processor 23, and outputs the image data to the image display section 26. The image display section 26 displays an image on the basis of the image data inputted from the image data generator 25. In this case, the person to be evaluated solves questions while watching the image displayed on the image display section 26.

The signal processor 23 outputs a request for information acquisition to the biological sensor 41. The request for information acquisition is for causing the biological sensor 41 to acquire at least one piece of information of the biological information or the action information in response to the input of the request for information acquisition, and output the at least one piece of information to the signal processor 23.

Upon acquiring information (at least one piece of information of the biological information or the action information) from the biological sensor 41, the signal processor 23 derives the arousal level 24e on the basis of the acquired information. The signal processor 23 derives the feature amount 24f corresponding to the classification index 24c on the basis of the derived arousal level 24e. The signal processor 23 selects one classification from among the plurality of classifications (1) to (4) in accordance with magnitude of the derived feature amount 24f (e.g., the lengths of the duration Δt1 and the rise time Δt2). The signal processor 23 evaluates the person to be evaluated on the basis of the selected classification (classification result 24g). The signal processor 23 stores, for example, the evaluation result 24h of the person to be evaluated in the storage section 24.

The image data generator 25 generates image data in which the classification index 24c and the feature amount 24f derived for evaluation are associated with each other. The image data generator 25 generates image data in which the classification index 24c and the time series data of the arousal level 24e are associated with each other. The image data generator 25 outputs the generated image data to the image display section 26. The image display section 26 displays an image based on the image data inputted from the image data generator 25. The image display section 26 displays, for example, an image as illustrated in FIGS. 5 to 7 on the display screen 26A.

### Effects

Next, description is given of effects of the biological information processing system 110.

In the present embodiment, as with the embodiment described above, the arousal level 24e is classified on the basis of the predetermined classification index 24c. This makes it possible to classify the person to be evaluated with use of the arousal level 24e that is objective data. As a result, for example, in a case of recruitment, it is possible to determine, from the arousal level 24e of the person to be evaluated, whether or not the person to be evaluated is a desired talent. In addition, for example, in a case of determining project members, it is possible to determine, from the arousal levels 24e of a large number of persons to be evaluated, whether or not the persons to be evaluated are members suitable to constitute the specific group. It is therefore possible to reduce mismatches.

In the present embodiment, the derived arousal level 24e is stored, in the storage section 24, in association with the identifier 24d of the person to be evaluated. This makes it possible to classify the person to be evaluated with use of the arousal level 24e that is objective data. As a result, for example, in a case of recruitment, it is possible to determine, from the arousal level 24e of the person to be evaluated, whether or not the person to be evaluated is a desired talent. In addition, for example, in a case of determining project members, it is possible to determine, from the arousal levels 24e of a large number of persons to be evaluated, whether or not the persons to be evaluated are members suitable to constitute the specific group. It is therefore possible to reduce mismatches.

### 4. Third Embodiment

[Configuration]

Next, description is given of an information processing system 120 according to a third embodiment of the present disclosure. FIG. 12 illustrates a schematic configuration example of the information processing system 120. The information processing system 120 is an objective evaluation system that evaluates a plurality of target living bodies on the basis of at least one of biological information or action information acquired from the plurality of target living bodies. In the present embodiment, the target living body is a human. It is to be noted that, in the information processing system 120, the target living body is not limited to the human.

The information processing system 120 includes an electronic apparatus 50 and a plurality of electronic apparatuses 60. The electronic apparatus 50 and each of the electronic apparatuses 60 are coupled to each other to enable data transmission/reception via a network 70. The information processing system 120 further includes a plurality of biological sensors 10. The plurality of biological sensors 10 are allocated one to each of the electronic apparatuses 60, and each of the biological sensors 10 is coupled to the electronic apparatus 60. The network 70 is a wireless or wired communication means, and examples thereof include the Internet, a WAN (Wide Area Network), a LAN (Local Area Network), a public communication network, a dedicated line, and the like.

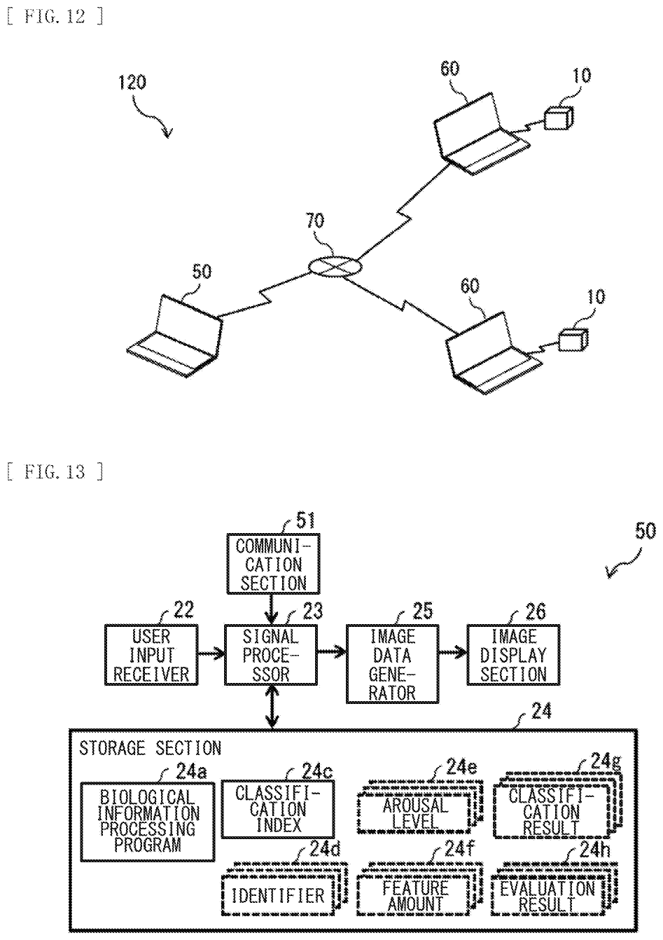

The electronic apparatus 50 includes, for example, a communication section 51, the user input receiver 22, the signal processor 23, the storage section 24, the image data generator 25, and the image display section 26, as illustrated in FIG. 13. The communication section 51 includes, for example, an interface that is able to communicate with each of the electronic apparatuses 60 via the network 70. The signal processor 23 receives detection information 65b that includes at least one of biological information or action information, and the identifier 24d of the person to be evaluated from each of the electronic apparatuses 60 via the communication section 51. The signal processor 23 derives the arousal level 24e of the person to be evaluated on the basis of the received detection information 65b. In this case, the signal processor 23 derives the arousal level 24e of the person to be evaluated with use of one method of various methods described above. The signal processor 23 derives, for example, time series data as the arousal level 24e. For example, the signal processor 23 further stores, in the storage section 24, the derived time series data in association with the received identifier 24d.

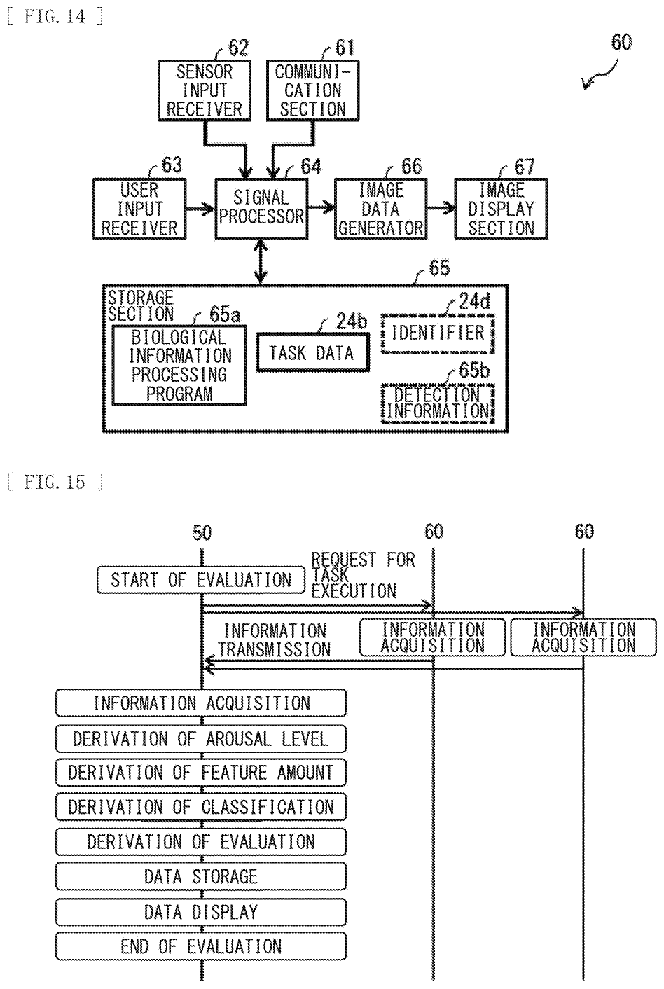

The electronic apparatuses 60 each include, for example, a communication section 61, a sensor input receiver 62, a user input receiver 63, a signal processor 64, a storage section 65, an image data generator 66, and an image display section 67, as illustrated in FIG. 14.

The communication section 61 includes, for example, an interface that is able to communicate with the electronic apparatus 50 via the network 70. The sensor input receiver 62 receives an input from the biological sensor 10, and outputs the input to the signal processor 64. The input from the biological sensor 10 includes at least one (detection information 65b) of the biological information or the action information. The sensor input receiver 62 includes, for example, an interface that is able to communicate with the biological sensor 10. The user input receiver 63 receives an input from a user, and outputs the input to the signal processor 64. Examples of the input from the user include attribute information (e.g., a name or the like) about the person to be evaluated, and an evaluation start instruction. The user input receiver 63 includes, for example, an input interface such as a keyboard, a mouse, or a touch panel.

The storage section 65 is, for example, a volatile memory such as a DRAM, or a non-volatile memory such as an EEPROM or a flash memory. A biological information processing program 65a, and the task data 24b that is used in the biological information processing program 65a are stored in the storage section 65. The biological information processing program 65a includes a series of procedures for acquiring the detection information 65b. Furthermore, the identifier 24d obtained by processing by the biological information processing program 65a is stored in the storage section 65.

The signal processor 64 includes, for example, a processor. The signal processor 64 executes the biological information processing program 65a stored in the storage section 65. A function of the signal processor 64 is implemented, for example, by executing the biological information processing program 65a by the signal processor 64. The signal processor 64 executes processing in a series of procedures to acquire the detection information 65b. The signal processor 64 reads, for example, a predetermined plurality of pieces of question data from the task data 24b, and sequentially outputs the read plurality of pieces of question data to the image data generator 66. The image data generator 66 generates image data including the pieces of question data inputted from the signal processor 64, and outputs the image data to the image display section 67. The image display section 67 displays an image on the basis of the image data inputted from the image data generator 66. The person to be evaluated solves questions while watching the image displayed on the image display section 67.

The person to be evaluated inputs, for example, an answer corresponding to a piece of question data to the user input receiver 63. For example, upon acquiring an answer corresponding to a question displayed on the image display section 67 from the user input receiver 63, the signal processor 64 outputs the next piece of question data to the image data generator 66. It is to be noted that the person to be evaluated may write an answer corresponding to a piece of question data on a paper sheet and input a notification that answering is completed to the user input receiver 63. In this case, for example, upon receiving the notification that answering is completed from the user input receiver 63, the signal processor 64 outputs the next piece of question data to the image data generator 66. The person to be evaluated may write an answer corresponding to a piece of question data on a paper sheet and input nothing to the user input receiver 63. In this case, the signal processor 64 outputs the next piece of question data to the image data generator 66 at regular intervals, for example. The signal processor 64 transmits, to the electronic apparatus 50 via the communication section 61, the detection information 65b acquired from the person to be evaluated while the person to be evaluated is performing a task (specific task) of solving a plurality of questions, together with the identifier 24d of the person to be evaluated.

The information processing system 120 may evaluate an applicant (person to be evaluated) for recruitment. In this case, upon obtaining the arousal level 24e, the signal processor 23 stores, in the storage section 24, the classification result 24g derived from the obtained arousal level 24e in association with the identifier 24d of the person to be evaluated. In a case where the classification result 24g stored in the storage section 24 meets the recruitment criteria, the signal processor 23 stores, in the storage section 24, the identifier 24d of a person who meets the recruitment criteria as the evaluation result 24h.

The information processing system 120 may evaluate a plurality of persons to be evaluated to select persons suitable to constitute a specific group (e.g., a team in an organization). In this case, every time the signal processor 23 obtains the arousal level 24e from the person to be evaluated, the signal processor 23 stores, in the storage section 24, the classification result 24g derived from the obtained arousal level 24e in association with the identifier 24d of the person to be evaluated. The signal processor 23 selects a plurality of identifiers 24d suitable to constitute the specific group (e.g., the team in the organization) on the basis of the respective classification results 24g of the plurality of persons to be evaluated as evaluation targets stored in the storage section 24. The signal processor 23 extracts the classification results 24g that meets criteria for constituting the specific group (e.g., the team in the organization) from among a plurality of classification results 24g, corresponding to the plurality of persons to be evaluated, stored in the storage section 24. The signal processor 23 stores, in the storage section 24, a plurality of identifiers 24d corresponding to a plurality of extracted classification results 24g as the evaluation results 24h.

In a case where a plurality of persons to be evaluated are evaluated to select persons suitable to constitute the specific group (e.g., the team in the organization), the image data generator 25 generates image data in which the classification index 24c and a plurality of feature amounts 24f derived for evaluation of the plurality of persons to be evaluated are associated with each other. The image data generator 25 generates image data in which the classification index 24c and pieces of time series data of a plurality of arousal levels 24e of the persons to be evaluated are associated with each other. The image data generator 25 outputs the generated image data to the image display section 26.

The image display section 26 displays an image based on the image data inputted from the image data generator 25. In this case, the image display section 26 displays, for example, an image as illustrated in FIGS. 6 and 7 on the display screen 26A. On the display screen 26A, for example, as illustrated in FIGS. 6 and 7, the classification indices 24c are displayed in the form of a two-dimensional graph, and the plurality of feature amounts 24f is displayed as plots in one or a plurality of quadrants of the two-dimensional graph. For example, as illustrated in FIGS. 6 and 7, the pieces of time series data of a plurality of arousal levels 24e are displayed on the display screen 26A to be aligned in time and superimposed on each other.

When the pieces of time series data of the plurality of arousal levels 24e are nearly synchronized with each other as illustrated in FIG. 6, a plurality of persons to be evaluated of which the arousal levels 24e have been calculated may be classified into a common classification index 24c. Meanwhile, as illustrated in FIG. 7, when the pieces of time series data of the plurality of arousal levels 24e are not synchronized with each other at all, a plurality of persons to be evaluated of which the arousal levels 24e have been calculated may be classified into classification indices 24c different from each other. Accordingly, the user is able to evaluate a plurality of persons to be evaluated of which the arousal levels 24e have been calculated from synchronization of the pieces of time series data of the plurality of arousal levels 24e displayed on the image display section 26. When an image (the pieces of time series data of the plurality of arousal levels 24e) as illustrated in FIGS. 6 and 7 is displayed on the image display section 26, the signal processor 23 receives selection of a plurality of arousal levels 24e from among the plurality of arousal levels 24e or selection of a plurality of identifiers 24d from among the plurality of identifiers 24d via the user input receiver 22. The signal processor 23 selects a plurality of identifiers 24d suitable to constitute the specific group (e.g., the team in the organization) on the basis of received contents (selection result). The signal processor 23 stores, in the storage section 24, the selected plurality of identifiers 24d as the evaluation result 24h. Thus, the user is able to evaluate a plurality of persons to be evaluated of which the arousal levels 24e have been calculated from synchronization of the pieces of time series data of the plurality of arousal levels 24e displayed on the image display section 26.

[Operation]

Next, description is given of an operation of the information processing system 120. FIG. 15 illustrates an example of an evaluation procedure in the information processing system 120.

First, the electronic apparatus 50 (signal processor 23) loads the biological information processing program 24a from the storage section 24, and starts execution of a series of procedures for evaluation described in the biological information processing program 24a. The electronic apparatus 60 (signal processor 64) loads the biological information processing program 65a from the storage section 65, and starts execution of a series of procedures for evaluation described in the biological information processing program 65a.

The electronic apparatus 50 (signal processor 23) transmits a request for task execution to each of the electronic apparatuses 60 via the communication section 51. Upon inputting the request for task execution to the electronic apparatus 60 (signal processor 64), the electronic apparatus 60 (signal processor 64) reads a predetermined plurality of pieces of question data from the task data 24b, and sequentially outputs the read plurality of pieces of question data to the image data generator 66. The image data generator 66 generates image data including the pieces of question data inputted from the signal processor 64, and outputs the image data to the image display section 67. The image display section 67 displays an image on the basis of the image data inputted from the image data generator 66. In this case, the person to be evaluated solves questions while watching the image displayed on the image display section 67.

The electronic apparatus 60 (signal processor 64) acquires the detection information 65b about the person to be evaluated from the biological sensor 10 while the person to be evaluated is performing a task (specific task) of solving a plurality of questions. Upon acquiring the detection information 65b from the biological sensor 10, the electronic apparatus 60 (signal processor 64) transmits the detection information 65b and the identifier 24d of the person to be evaluated to the electronic apparatus 50 via the communication section 61.

Upon acquiring the detection information 65b and the identifier 24d of the person to be evaluated from each of the electronic apparatuses 50 via the communication section 61, the electronic apparatus 50 (signal processor 23) derives the arousal level 24e for each person to be evaluated on the basis of the acquired information. The electronic apparatus 50 (signal processor 23) derives the feature amount 24f corresponding to the classification index 24c for each person to be evaluated on the basis of the derived arousal level 24e. The signal processor 23 selects, for each person to be evaluated, one classification from among the plurality of classifications (1) to (4) in accordance with magnitude of the derived feature amount 24f (e.g., the lengths of the duration Δt1 and the rise time Δt2). The signal processor 23 evaluates the person to be evaluated, on the basis of the selected classification (classification result 24g). The signal processor 23 stores, in the storage section 24, for example, the evaluation result 24h for each person to be evaluated.

The image data generator 25 generates image data in which the classification index 24c and the feature amount 24f derived for evaluation are associated with each other. The image data generator 25 generates image data in which the classification index 24c and time series data of the arousal level 24e that are generated for each person to be evaluated are associated with each other. The image data generator 25 outputs the generated image data to the image display section 26. The image display section 26 displays an image based on the image data inputted from the image data generator 25. The image display section 26 displays, for example, an image as illustrated in FIGS. 6 and 7 on the display screen 26A.

### Effects

Next, description is given of effects of the information processing system 120.

In the present embodiment, as with the first and second embodiments described above and modification examples thereof, the arousal level 24e is classified on the basis of the predetermined classification index 24c. This makes it possible to classify the person to be evaluated with use of the arousal level 24e that is objective data. As a result, for example, in a case of determining project members, it is possible to determine, from the arousal levels 24e of a large number of persons to be evaluated, whether or not the persons to be evaluated are members suitable to constitute the specific group. It is therefore possible to reduce mismatches.

In the present embodiment, the derived arousal level 24e is stored, in the storage section 24, in association with the identifier 24d of the person to be evaluated. This make it possible to classify the person to be evaluated with use of the arousal level 24e that is objective data. As a result, for example, in a case of determining project members, it is possible to determine, from the arousal levels 24e of a large number of persons to be evaluated, whether or not the persons to be evaluated are members suitable to constitute the specific group. It is therefore possible to reduce mismatches.

### 5. Fourth Embodiment

[Configuration]

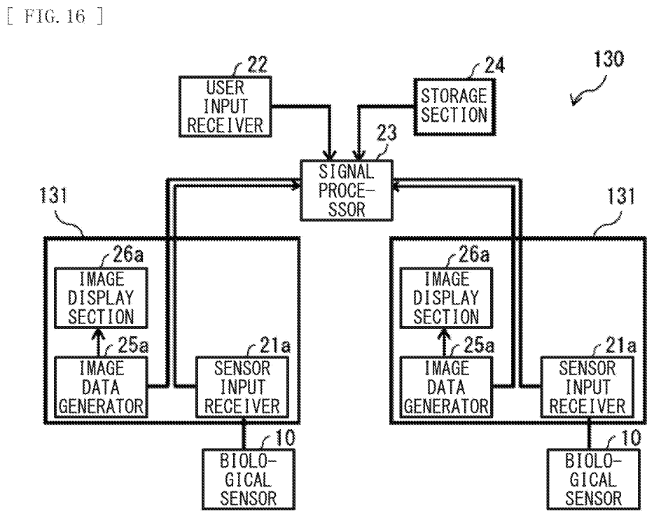

Next, description is given of an information processing device 130 according to a fourth embodiment of the present disclosure. FIG. 16 illustrates a schematic configuration example of the information processing device 130. The information processing device 130 is an objective evaluation system that evaluates a plurality of target living bodies on the basis of at least one of biological information or action information acquired from the plurality of target living bodies. In the present embodiment, the target living body is a human. It is to be noted that, in the information processing device 130, the target living body is not limited to the human.

The information processing device 130 includes a plurality of (e.g., two) devices 131, the signal processor 23 coupled to the plurality of (e.g., two) devices, the user input receiver 22, and the storage section 24. Each of the devices 131 is, for example, a device such as eyeglasses, and executes, by control by the signal processor 23, an operation similar to those of the electronic apparatuses 20, 40, and 50, and the information processing system 120 according to the first to fourth embodiments described above and modification examples thereof. In other words, in the present embodiment, one information processing device 130 is shared by a plurality of users.

Each of the devices 131 includes, for example, a sensor input receiver 21a, an image data generator 25a, and an image display section 26a. For example, one biological sensor 10 is mounted on each of the devices 131.

In the present embodiment, as with the first and second embodiments described above and the modification examples thereof, emotional information 16c about the target living body is estimated on the basis of information (at least one of biological information or motion information) about the target living body acquired by the biological sensor 10, and is displayed on a display surface of the image display section 26a.

In the present embodiment, as with the first and second embodiments described above and the modification examples thereof, the arousal level 24e is classified on the basis of the predetermined classification index 24c. This makes it possible to classify the person to be evaluated with use of the arousal level 24e that is objective data. As a result, for example, in a case of determining project members, it is possible to determine, from the arousal levels 24e of a large number of persons to be evaluated, whether or not the persons to be evaluated are members suitable to constitute the specific group. It is therefore possible to reduce mismatches.

In the present embodiment, the derived arousal level 24e is stored, in the storage section 24, in association with the identifier 24d of the person to be evaluated. This makes it possible to classify the person to be evaluated with use of the arousal level 24e that is objective data. As a result, for example, in a case of determining project members, it is possible to determine, from the arousal levels 24e of a large number of persons to be evaluated, whether or not the persons to be evaluated are members suitable to constitute the specific group. It is therefore possible to reduce mismatches.

### 4. Modification Examples of Respective Embodiments

Next, description is given of modification examples of the biological information processing systems 100 and 110, the information processing system 120, and the information processing device 130.

### Modification Example A

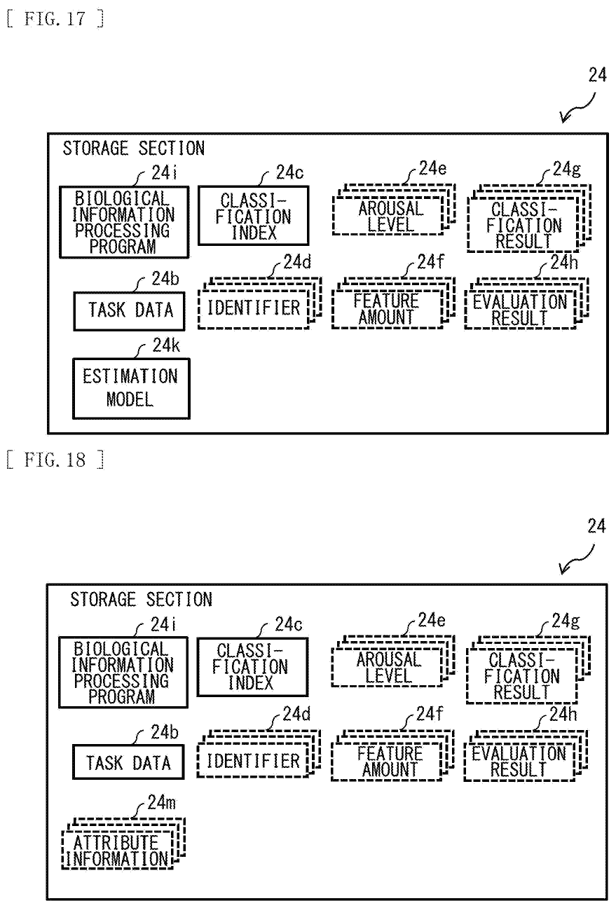

In the first to fourth embodiments described above, for example, the storage section 24 may include an estimation model 24k that estimates the arousal level 24e, as illustrated in FIG. 17. The estimation model 24k estimates the arousal level 24e on the basis of information (at least one piece of information of the biological information or the action information) acquired from the biological sensor 10. The estimation model 24k is, for example, the estimation model described in <1. About Arousal Level>. In this case, the storage section 24 includes a biological information processing program 24i in place of the biological information processing program 24a. The biological information processing program 24i implements a function (a series of processing procedures) of a portion, excluding the function of the estimation model 24k, of the biological information processing program 24a. Using the estimation model 24k in such a manner makes it possible to estimate the arousal level 24e with higher accuracy. As a result, it is possible to further reduce mismatches.

### Modification Example B

In addition, in the first to fourth embodiments described above and the modification examples thereof, for example, attribute information 24m may be stored in the storage section 24, as illustrated in FIG. 18. Examples of the attribute information 24m include attribute information such as the age, gender, and educational background of the person to be evaluated. In the present modification example, the signal processor 23 may evaluate the person to be evaluated, for example, with use of not only the feature amount 24f but also the attribute information 24m. Evaluating the person to be evaluated with use of not only the feature amount 24f but also the attribute information 24m in such a manner makes it possible to estimate the arousal level 24e with higher accuracy. As a result, it is possible to further reduce mismatches.

### Modification Example C

In addition, in the first to fourth embodiments described above and the modification examples thereof, the electronic apparatuses 20, 40, and 50, and the information processing device 130 may be coupled to a server apparatus by an external network. In this case, a server apparatus may include a program or an estimation model for executing a series of processing to estimate the arousal level 24e. In such a case, it is not necessary to provide the program or the estimation model for executing a series of processing to estimate the arousal level 24e in the electronic apparatuses 20, 40, and 50, and the information processing device 130. As a result, it is possible to share the program or the estimation model for executing a series of processing to estimate the arousal level 24e provided in the server apparatus by a plurality of electronic apparatuses 20, a plurality of electronic apparatuses 40, a plurality of electronic apparatuses 50, or a plurality of information processing devices 130.

### Modification Example D

In addition, in the first to fourth embodiments described above and the modification examples thereof, comfort/discomfort may be used in place of or together with the arousal level 24e. As with the arousal level 24e, the comfort/discomfort is a kind of emotional information. In the present modification example, a period in which comfort is kept may be used in place of duration Δt1 or together with the duration Δt1. In addition, in the present modification example, an index related to quickness of comfort/discomfort switching may be used in place of the rise time Δt2 or together with the rise time Δt2.

In the present modification example, at least one of the arousal level 24e or comfort/discomfort is classified on the basis of a predetermined classification index 24c. This makes it possible to classify the person to be evaluated with use of the arousal level 24e or the comfort/discomfort that is objective data. As a result, for example, in a case of recruitment, it is possible to determine, from the arousal level 24e or the comfort/discomfort of the person to be evaluated, whether or not the person to be evaluated is a desired talent. In addition, for example, in a case of determining project members, it is possible to determine, from the arousal levels 24e or the comfort/discomfort of a large number of persons to be evaluated, whether or not the persons to be evaluated are members suitable to constitute the specific group. It is therefore possible to reduce mismatches.

### Modification Example E

In the first embodiment described above and the modification examples thereof, for example, some functions of the electronic apparatus 20 may be provided in an external apparatus (e.g., a sever apparatus) different from the electronic apparatus 20. In this case, the electronic apparatus 20 and the external apparatus (e.g., the server apparatus) may be coupled to each other by any network, for example.

In addition, in the second embodiment described above and the modification examples thereof, some functions of the electronic apparatus 40 may be provided in an external apparatus (e.g., a server apparatus) different from the electronic apparatus 40. In this case, the electronic apparatus 40 and the external apparatus (e.g., a server apparatus) may be coupled to each other by any network, for example.

In addition, in the third embodiment described above and the modification examples thereof, for example, some functions of the electronic apparatus 50 may be provided in an external apparatus (e.g., a server apparatus) different from the electronic apparatus 50. In this case, the electronic apparatus 50 and the external apparatus (e.g., the server apparatus) may be coupled to each other by any network, for example.

In addition, in the fourth embodiment described above and the modification examples thereof, for example, some functions of the information processing device 130 may be provided in an external apparatus (e.g., a server apparatus) different from the information processing device 130. In this case, the information processing device 130 and the external apparatus (e.g., the server apparatus) may be coupled to each other by any network, for example.

### Modification Example F

In the first embodiment described above and the modification examples thereof, the electronic apparatus 20 and the biological sensor 10 may be coupled to each other by means other than the network 30.

### Modification Example G

In the first to fourth embodiments described above and the modification examples thereof, the biological sensor 10 may be mounted in a head-mounted display (HMD) 200, for example, as illustrated in FIG. 29. In the head-mounted display 200, for example, a detection electrode 203 of the biological sensor 10 may be provided on an inner surface or the like of each of a pad part 201 and a band part 202.

In addition, in the first to fourth embodiments described above and the modification examples thereof, the biological sensor 10 may be mounted, for example, in a head band 300 as illustrated in FIG. 30. In the head band 300, for example, a detection electrode 303 of the biological sensor 10 may be provided on an inner surface or the like of each of band parts 301 and 302 to be in contact with the head.

In addition, in the first to fourth embodiments described above and the modification examples thereof, the biological sensor 10 may be mounted, for example, in a headphone 400 as illustrated in FIG. 31. In the headphone 400, for example, a detection electrode 403 of the biological sensor 10 may be provided on an inner surface of a band part 401, an ear pad 402, or the like to be in contact with the head.

In addition, in the first to fourth embodiments described above and the modification examples thereof, the biological sensor 10 may be mounted, for example, in an earphone 500 as illustrated in FIG. 32. In the earphone 500, for example, a detection electrode 502 of the biological sensor 10 may be provided in an ear piece 501 to be inserted into the ear.

In addition, in the first to fourth embodiments described above and the modification examples thereof, the biological sensor 10 may be mounted, for example, in a watch 600 as illustrated in FIG. 33. In the watch 600, for example, a detection electrode 604 of the biological sensor 10 may be provided on an inner surface of a display part 601 that displays time or the like, an inner surface of a band part 602 (e.g., an inner surface of a buckle part 603), or the like.

In addition, in the first to fourth embodiments described above and the modification examples thereof, the biological sensor 10 may be mounted, for example, in glasses 700 as illustrated in FIG. 34. In the glasses 700, for example, a detection electrode 702 of the biological sensor 10 may be provided on an inner surface of a temple 701 or the like.

In addition, in the first to fourth embodiments described above and the modification examples thereof, the biological sensor 10 may also be mounted, for example, in a glove, a ring, a pencil, a pen, a controller of a game machine, or the like.

### Modification Example H

In the first to fourth embodiments described above and the modification examples thereof, the signal processor 23 may derive, for example, feature amounts as given below, on the basis of electric signals of a pulse wave, an electrocardiogram, and a blood flow of the person to be evaluated obtained by a sensor, and may derive the arousal level 24e of the person to be evaluated, on the basis of the derived feature amounts.

(Pulse Wave, Electrocardiogram, and Blood Flow)

It is possible to derive the arousal level 24e of the person to be evaluated by using, for example, feature amounts as given below, which are obtained on the basis of electric signals of a pulse wave, an electrocardiogram, and a blood flow obtained by the sensor.
Heart rate per 1 s
Average value of heart rates per 1 s within a predetermined period (window)
rmssd (root mean square successive difference): root mean square of successive heartbeat intervals
pnn50 (percentage of adjacent normal-to-normal intervals): percentage of the number of successive heartbeat intervals exceeding 50 ms
LF: area of PSD of heartbeat intervals between 0.04 Hz and 0.15 Hz
HF: area of PSD of heartbeat intervals between 0.15 Hz and 0.4 Hz
LF/(LF+HF)
HF/(LF+HF)
LF/HF
Heart rate entropy
SD1: standard deviation of Poincare plot (scatter diagram with an x-axis for t-th heartbeat interval and a y-axis for t+1-th heartbeat interval) in a direction of an axis of y=x
SD2: standard deviation of Poincare plot in a direction of an axis perpendicular to y=x
SD1/SD2
SDRR (standard deviation of RR interval): standard deviation of heartbeat interval

In addition, in the first to fourth embodiments described above and the modification examples thereof, the signal processor 23 may derive, for example, feature amounts as given below, on the basis of electric signals (EDA: electrodermal activity) of emotional sweating of the person to be evaluated obtained by the sensor, and may derive the arousal level 24e of the person to be evaluated, on the basis of the derived feature amounts.

(Emotional Sweating)

It is possible to derive the arousal level 24e of the person to be evaluated by using the feature amounts as given below, which are obtained on the basis of electric signals of emotional sweating obtained by the sensor.
Number of SCRs (skin conductance response) occurring per minute
Amplitude of SCR
Value of SCL (skin conductance level)
Rate of change of SCL

For example, it is possible to separate SCR and SCL from EDA by using a method described in the following literature:
Benedek, M., & Kaernbach, C. (2010). A continuous measure of phasic electrodermal activity. Journal of neuroscience methods, 190(1), 80-91.

It is to be noted that, in derivation of the arousal level 24e, a single modal (one physiological index) may be used, or a combination of a plurality of modals (a plurality of physiological indices) may be used.

The signal processor 23 derives the feature amounts described above by using, for example, regression formulae illustrated in FIGS. 35 to 42 to be described later.

FIG. 35 illustrates an example of a relationship between a task difference Δha [%] and the accuracy rate R [%]. The task difference Δha [%] is a task difference in the pnn50 of a pulse wave between the time of solving the high difficulty level questions and the time of solving the low difficulty level questions. The accuracy rate R [%] is the accuracy rate at the time of solving the high difficulty level questions. The task difference Δha is a vector volume obtained by subtracting the pnn50 of the pulse wave at the time of solving the low difficulty level questions from the pnn50 of the pulse wave at the time of solving the high difficulty level questions. In FIG. 35, data for respective users are plotted, and features of the entirety of users are represented by a regression formula (regression line). In FIG. 35, the regression formula is represented by R=a10×Δha+b10.

A small task difference Δha in the pnn50 of the pulse wave means that the difference in the pnn50 of the pulse wave between the time of solving the high difficulty level questions and the time of solving the low difficulty level questions is small. It can be said that a user who has obtained such a result has a tendency in which, as the difficulty level of the questions becomes high, a task difference in the pnn50 of the pulse wave becomes smaller as compared with other users. Meanwhile, a large task difference Δha in the pnn50 of the pulse wave means that the difference in the pnn50 of the pulse wave between the time of solving the high difficulty level questions and the time of solving the low difficulty level questions is large. It can be said that a user who has obtained such a result has a tendency in which, as the difficulty level of the questions becomes high, the task difference in the pnn50 of the pulse wave becomes larger as compared with other users.

It is appreciated from FIG. 35 that, when the task difference Δha in the pnn50 of the pulse wave is large, the accuracy rate R for questions becomes high, and that, when the task difference Δha in the pnn50 of the pulse wave is small, the accuracy rate R for questions becomes small. It is appreciated from the above that a person who has large pnn50 of the pulse wave for difficult questions tends to have a high accuracy rate R (i.e., be able to answer accurately even for difficult questions to the same degree as for simple questions). Conversely, it is appreciated that a person who has small pnn50 of the pulse wave even for difficult questions tends to have a low accuracy rate R (i.e., the accuracy rate for the difficult questions is lowered).

Herein, as described above, it can be seen from FIG. 28 that, when the accuracy rate is high, the arousal level is low, and when the accuracy rate is low, the arousal level is high. It can be inferred from the above that, when the task difference Δha in the pnn50 of the pulse wave is large, the arousal level of the user is lower than the predetermined standard. in addition, it can be inferred that, when the task difference Δha in the pnn50 of the pulse wave is small, the arousal level of the user is higher than the predetermined standard.

It is appreciated from the above that using the task difference Δha in the pnn50 of the pulse wave and the regression formulae in FIGS. 28 and 35 makes it possible to derive the arousal level of the user.

FIG. 36 illustrates an example of a relationship between a task difference Δhb [%] and the accuracy rate R [%]. The task difference Δhb [%] is a task difference in dispersion of the pnn50 of the pulse wave between the time of solving the high difficulty level questions and the time of solving the low difficulty level questions. The accuracy rate R [%] is the accuracy rate at the time of solving the high difficulty level questions. The task difference Δhb is a vector volume obtained by subtracting the dispersion of the pnn50 of the pulse wave at the time of solving the low difficulty level questions from the dispersion of the pnn50 of the pulse wave at the time of solving the high difficulty level questions. In FIG. 36, data for respective users are plotted, and features of the entirety of users are represented by a regression formula (regression line). In FIG. 36, the regression formula is represented by R=all x Δhb+b11.

A small task difference Δhb in the dispersion of the pnn50 of the pulse wave means that the difference in the dispersion of the pnn50 of the pulse wave between the time of solving the high difficulty level questions and the time of solving the low difficulty level questions is small. It can be said that a user who has obtained such a result has a tendency in which, as the difficulty level of the questions becomes high, the task difference in the dispersion of the pnn50 of the pulse wave becomes smaller as compared with other users. Meanwhile, a large task difference Δhb in the dispersion of the pnn50 of the pulse wave means that the difference in the dispersion of the pnn50 of the pulse wave between the time of solving the high difficulty level questions and the time of solving the low difficulty level questions is large. It can be said that a user who has obtained such a result has a tendency in which, as the difficulty level of the questions becomes high, the task difference in the dispersion of the pnn50 of the pulse wave becomes larger as compared with other users.

It is appreciated from FIG. 36 that, when the task difference Δhb in the dispersion of the pnn50 of the pulse wave is large, the accuracy rate R for questions becomes high, and that, when the task difference Δhb in the dispersion of the pnn50 of the pulse wave is small, the accuracy rate R for questions becomes small. It is appreciated from the above that a person who has large dispersion of the pnn50 of the pulse wave for difficult questions tends to have a high accuracy rate R (i.e., be able to answer accurately even for difficult questions to the same degree as for simple questions). Conversely, it is appreciated that a person who has small dispersion of the pnn50 of the pulse wave even for difficult questions tends to have a low accuracy rate R (i.e., the accuracy rate for the difficult questions is lowered).

Herein, as described above, it can be seen from FIG. 28 that, when the accuracy rate is high, the arousal level is low, and when the accuracy rate is low, the arousal level is high. It can be inferred from the above that, when the task difference Δhb in the dispersion of the pnn50 of the pulse wave is large, the arousal level of the user is lower than the predetermined standard. In addition, it can be inferred that, when the task difference Δha in the dispersion of the pnn50 of the pulse wave is small, the arousal level of the user is higher than the predetermined standard.

It is appreciated from the above that using the task difference Δhb in the dispersion of the pnn50 of the pulse wave and the regression formulae in FIGS. 28 and 36 makes it possible to derive the arousal level of the user.

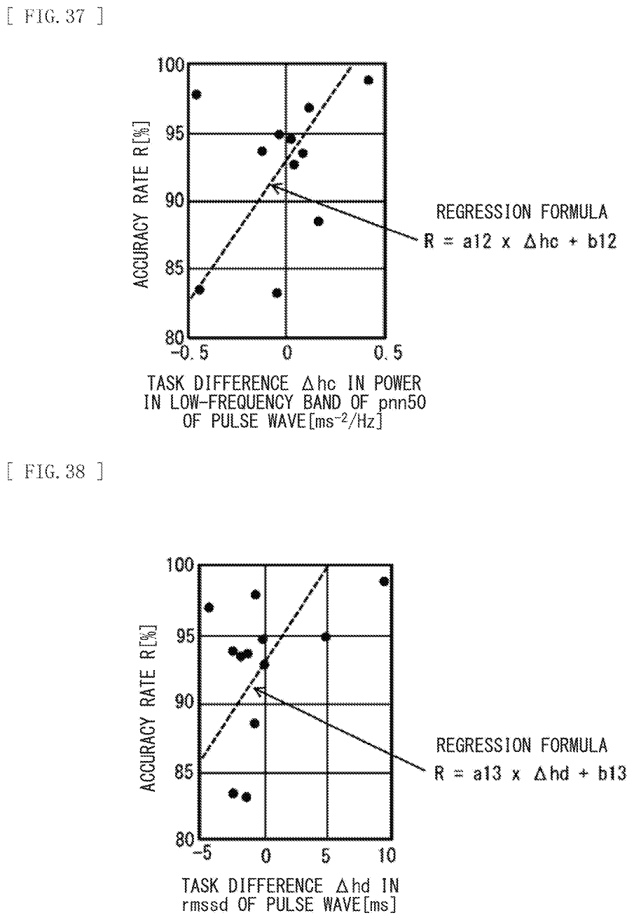

FIG. 37 illustrates an example of a relationship between a task difference Δhc [ms' Hz] and the accuracy rate R [%]. The task difference Δhc [ms' Hz] is a task difference in power in a low-frequency band (around 0.01 Hz) of a power spectrum obtained by performing FFT on the pnn50 of the pulse wave between the time of solving the high difficulty level questions and the time of solving the low difficulty level questions. The accuracy rate R [%] is the accuracy rate at the time of solving the high difficulty level questions. Hereinafter, the “power in a low-frequency band (around 0.01 Hz) of a power spectrum obtained by performing FFT on the pnn50 of the pulse wave” is referred to as “power in the low-frequency band of the pnn50 of the pulse wave”. The task difference Δhc is a vector volume obtained by subtracting the power in the low-frequency band of the pnn50 of the pulse wave at the time of solving the low difficulty level questions from the power in the low-frequency band of the pnn50 of the pulse wave at the time of solving the high difficulty level questions. In FIG. 37, data for respective users are plotted, and features of the entirety of users are represented by a regression formula (regression line). In FIG. 37, the regression formula is represented by R=a12×Δhc+b12.

A large task difference Δhc in the power in the low-frequency band of the pnn50 of the pulse wave means that the difference in the power in the low-frequency band of the pnn50 of the pulse wave between the time of solving the high difficulty level questions and the time of solving the low difficulty level questions is large. It can be said that a user who has obtained such a result has a tendency in which, when solving the high difficulty level questions, the task difference in the power in the low-frequency band of the pnn50 of the pulse wave becomes larger as compared with other users. Meanwhile, a small task difference Δhc in the power in the low-frequency band of the pnn50 of the pulse wave means that the difference in the power in the low-frequency band of the pnn50 of the pulse wave between the time of solving the high difficulty level questions and the time of solving the low difficulty level questions is small. It can be said that a user who has obtained such a result has a tendency in which, as the difficulty level of the questions becomes high, the task difference in the power in the low-frequency band of the pnn50 of the pulse wave becomes smaller as compared with other users.

It is appreciated from FIG. 37 that, when the task difference Δhc in the power in the low-frequency band of the pnn50 of the pulse wave is large, the accuracy rate R for questions becomes high, and that, when the task difference Δhc in the power in the low-frequency band of the pnn50 of the pulse wave is small, the accuracy rate R for questions becomes low. It is appreciated from the above that a person who has large power in the low-frequency band of the pnn50 of the pulse wave even for difficult questions tends to have a high accuracy rate R (i.e., be able to answer accurately even for difficult questions to the same degree as for simple questions). Conversely, it is appreciated that a person who has small power in the low-frequency band of the pnn50 of the pulse wave for difficult questions tends to have a low accuracy rate R (i.e., the accuracy rate for the difficult questions is lowered).

Herein, as described above, it can be seen from FIG. 28 that, when the accuracy rate is high, the arousal level is low, and when the accuracy rate is low, the arousal level is high. It can be inferred from the above that, when task difference Δhc in the power in the low-frequency band of the pnn50 of the pulse wave is small, the arousal level of the user is lower than the predetermined standard. In addition, it can be inferred that, when the task difference Δhc in the power in the low-frequency band of the pnn50 of the pulse wave is large in a negative direction, the arousal level of the user is higher than the predetermined standard.

It is appreciated from the above that using the task difference Δhc in the power in the low-frequency band of the pnn50 of the pulse wave and the regression formulae in FIGS. 28 and 37 makes it possible to derive the arousal level of the user.

FIG. 38 illustrates an example of a relationship between a task difference Δhd [ms] and the accuracy rate R [%]. The task difference Δhd [ms] is a task difference in the rmssd of the pulse wave between the time of solving the high difficulty level questions and the time of solving the low difficulty level questions. The accuracy rate R [%] is the accuracy rate at the time of solving the high difficulty level questions. The task difference Δhd is a vector volume obtained by subtracting the rmssd of the pulse wave at the time of solving the low difficulty level questions from the rmssd of the pulse wave at the time of solving the high difficulty level questions. In FIG. 38, data for respective users are plotted, and features of the entirety of users are represented by a regression formula (regression line). In FIG. 38, the regression formula is represented by R=a13×Δhd+b13.

A large task difference Δhd in the rmssd of the pulse wave means that the difference in the rmssd of the pulse wave between the time of solving the high difficulty level questions and the time of solving the low difficulty level questions is large. It can be said that a user who has obtained such a result has a tendency in which, when solving the high difficulty level questions, the task difference in the rmssd of the pulse wave becomes larger as compared with other users. Meanwhile, a small task difference Δhd in the rmssd of the pulse wave means that the difference in the rmssd of the pulse wave between the time of solving the high difficulty level questions and the time of solving the low difficulty level questions is small. It can be said that a user who has obtained such a result has a tendency in which, as the difficulty level of the questions becomes high, the task difference in the rmssd of the pulse wave becomes smaller as compared with other users.

It is appreciated from FIG. 38 that, when the task difference Δhd in the rmssd of the pulse wave is large, the accuracy rate R for questions becomes high, and that, when the task difference Δhd in the rmssd of the pulse wave is small, the accuracy rate R for questions becomes small. It is appreciated from the above that a person who has large rmssd of the pulse wave even for difficult questions tends to have a high accuracy rate R (i.e., be able to answer accurately even for difficult questions to the same degree as for simple questions). Conversely, it is appreciated that a person who has small rmssd of the pulse wave for difficult questions tends to have a low accuracy rate R (i.e., the accuracy rate for the difficult questions is lowered).

Herein, as described above, it can be seen from FIG. 28 that, when the accuracy rate is high, the arousal level is low, and when the accuracy rate is low, the arousal level is high. It can be inferred from the above that, when the task difference Δhd in the rmssd of the pulse wave is small, the arousal level of the user is lower than the predetermined standard. In addition, it can be inferred that, when the task difference Δhd in the rmssd of the pulse wave is large in the negative direction, the arousal level of the user is higher than the predetermined standard.

It is appreciated from the above that using the task difference Δhd in the rmssd of the pulse wave and the regression formulae in FIGS. 28 and 38 makes it possible to derive the arousal level of the user.

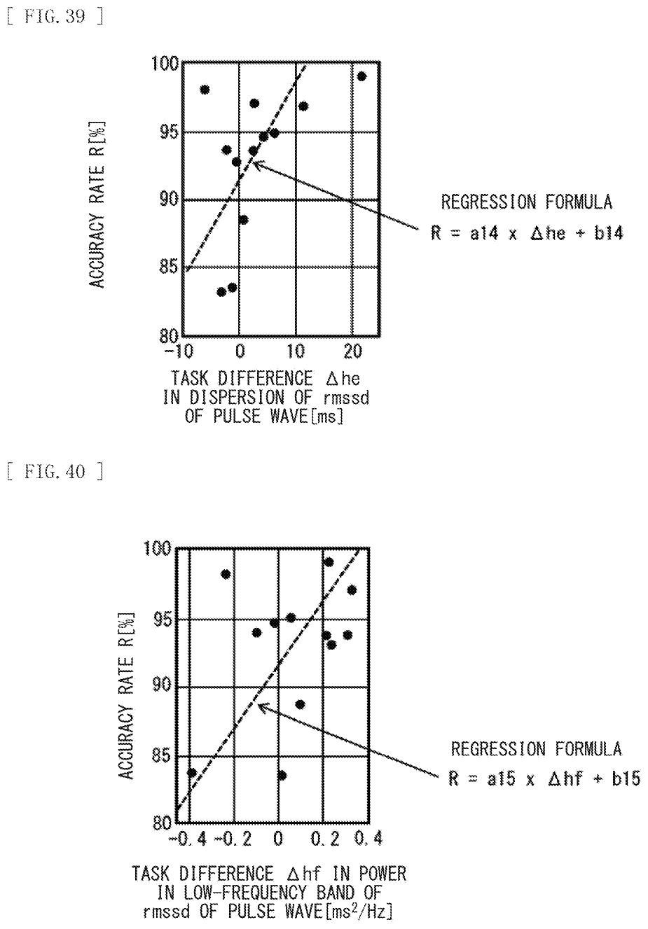

FIG. 39 illustrates an example of a relationship between a task difference Δhe [ms] and the accuracy rate R [%]. The task difference Δhe [ms] is a task difference in dispersion of the rmssd of the pulse wave between the time of solving the high difficulty level questions and the time of solving the low difficulty level questions. The accuracy rate R [%] is the accuracy rate at the time of solving the high difficulty level questions. The task difference Δhe is a vector volume obtained by subtracting the dispersion of the rmssd of the pulse wave at the time of solving the low difficulty level questions from the dispersion of the rmssd of the pulse wave at the time of solving the high difficulty level questions. In FIG. 39, data for respective users are plotted, and features of the entirety of users are represented by a regression formula (regression line). In FIG. 39, the regression formula is represented by R=a14×Δhe+b14.

A large task difference Δhe in the dispersion of the rmssd of the pulse wave means that the difference in the dispersion of the rmssd of the pulse wave between the time of solving the high difficulty level questions and the time of solving the low difficulty level questions is large. It can be said that a user who has obtained such a result has a tendency in which, when solving the high difficulty level questions, the task difference in the dispersion of the rmssd of the pulse wave becomes larger as compared with other users. Meanwhile, a small task difference Δhe in the dispersion of the rmssd of the pulse wave means that the difference in the dispersion of the rmssd of the pulse wave between the time of solving the high difficulty level questions and the time of solving the low difficulty level questions is small. It can be said that a user who has obtained such a result has a tendency in which, as the difficulty level of the questions becomes high, the task difference in the dispersion of the rmssd of the pulse wave becomes smaller as compared with other users.

It is appreciated from FIG. 39 that, when the task difference Δhe in the dispersion of the rmssd of the pulse wave is large, the accuracy rate R for questions becomes high, and that, when the task difference Δhe in the dispersion of the rmssd of the pulse wave is small, the accuracy rate R for questions becomes small. It is appreciated from the above that a person who has large dispersion of the rmssd of the pulse wave even for difficult questions tends to have a high accuracy rate R (i.e., be able to answer accurately even for difficult questions to the same degree as for simple questions). Conversely, it is appreciated that a person who has small dispersion of the rmssd of the pulse wave for difficult questions tends to have a low accuracy rate R (i.e., the accuracy rate for the difficult questions is lowered).

Herein, as described above, it can be seen from FIG. 28 that, when the accuracy rate is high, the arousal level is low, and when the accuracy rate is low, the arousal level is high. It can be inferred from the above that, when the task difference Δhe in the dispersion of the rmssd of the pulse wave is small, the arousal level of the user is lower than the predetermined standard. In addition, it can be inferred that, when the task difference Δhe in the dispersion of the rmssd of the pulse wave is large in the negative direction, the arousal level of the user is higher than the predetermined standard.

It is appreciated from the above that using the task difference Δhe in the dispersion of the rmssd of the pulse wave and the regression formulae in FIGS. 28 and 39 makes it possible to derive the arousal level of the user.

FIG. 40 illustrates an example of a relationship between a task difference Δhf [ms²/Hz] and the accuracy rate R [%]. The task difference Δhf [ms²/Hz] is a task difference in power in a low-frequency band (around 0.01 Hz) of a power spectrum obtained by performing FFT on the rmssd of the pulse wave between the time of solving the high difficulty level questions and the time of solving the low difficulty level questions. The accuracy rate R [%] is the accuracy rate at the time of solving the high difficulty level questions. Hereinafter, the “power in a low-frequency band (around 0.01 Hz) of a power spectrum obtained by performing FFT on the rmssd of the pulse wave” is referred to as “power in the low-frequency band of the rmssd of the pulse wave”. The task difference Δhf is a vector volume obtained by subtracting the power in the low-frequency band of the rmssd of the pulse wave at the time of solving the low difficulty level questions from the power in the low-frequency band of the rmssd of the pulse wave at the time of solving the high difficulty level questions. In FIG. 40, data for respective users are plotted, and features of the entirety of users are represented by a regression formula (regression line). In FIG. 40, the regression formula is represented by R=a15×Δhf+b15.

A large task difference Δhf in the power in the low-frequency band of the rmssd of the pulse wave means that the difference in the power in the low-frequency band of the rmssd of the pulse wave between the time of solving the high difficulty level questions and the time of solving the low difficulty level questions is large. It can be said that a user who has obtained such a result has a tendency in which, when solving the high difficulty level questions, the task difference in the power in the low-frequency band of the rmssd of the pulse wave becomes larger as compared with other users. Meanwhile, a small task difference Δhf in the power in the low-frequency band of the rmssd of the pulse wave means that the difference in the power in the low-frequency band of the rmssd of the pulse wave between the time of solving the high difficulty level questions and the time of solving the low difficulty level questions is small. It can be said that a user who has obtained such a result has a tendency in which, as the difficulty level of the questions becomes high, the task difference in the power in the low-frequency band of the rmssd of the pulse wave becomes smaller as compared with other users.

It is appreciated from FIG. 40 that, when the task difference Δhf in the power in the low-frequency band of the rmssd of the pulse wave is large, the accuracy rate R for questions becomes high, and that, when the task difference Δhf in the power in the low-frequency band of the rmssd of the pulse wave is small, the accuracy rate R for questions becomes small. It is appreciated from the above that a person who has large power in the low-frequency band of the rmssd of the pulse wave even for difficult questions tends to have a high accuracy rate R (i.e., be able to answer accurately even for difficult questions to the same degree as for simple questions). Conversely, it is appreciated that a person who has small power in the low-frequency band of the rmssd of the pulse wave for difficult questions tends to have a low accuracy rate R (i.e., the accuracy rate for the difficult questions is lowered).

Herein, as described above, it can be seen from FIG. 28 that, when the accuracy rate is high, the arousal level is low, and when the accuracy rate is low, the arousal level is high. It can be inferred from the above that, when the task difference Δhf in the power in the low-frequency band of the rmssd of the pulse wave is small, the arousal level of the user is lower than the predetermined standard. In addition, it can be inferred that, when the task difference Δhf in the power in the low-frequency band of the rmssd of the pulse wave is large in the negative direction, the arousal level of the user is higher than the predetermined standard.

It is appreciated from the above that using the task difference Δhf in the power in the low-frequency band of the rmssd of the pulse wave and the regression formulae in FIGS. 28 and 40 makes it possible to derive the arousal level of the user.

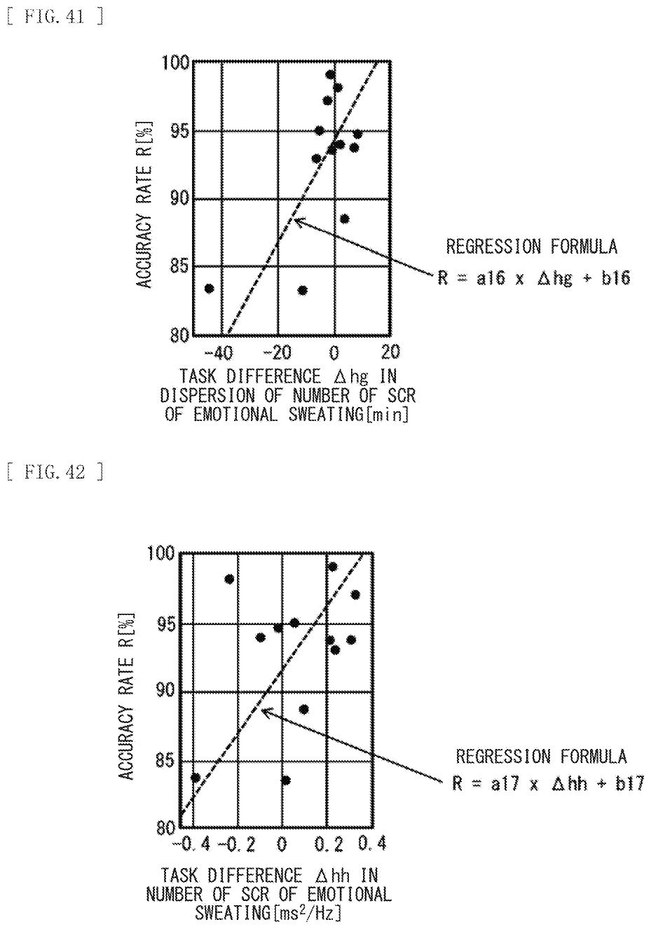

FIG. 41 illustrates an example of a relationship between a task difference Δhg [min] and the accuracy rate R [%]. The task difference Δhg [min] is a task difference in dispersion of the number of SCRs of the emotional sweating between the time of solving the high difficulty level questions and the time of solving the low difficulty level questions. The accuracy rate R [%] is the accuracy rate at the time of solving the high difficulty level questions. The task difference Δhg is a vector volume obtained by subtracting the dispersion of the number of the SCRs of the emotional sweating at the time of solving the low difficulty level questions from the dispersion of the number of SCRs of the emotional sweating at the time of solving the high difficulty level questions. In FIG. 41, data for respective users are plotted, and features of the entirety of users are represented by a regression formula (regression line). In FIG. 41, the regression formula is represented by R=a16×Δhg+b16.

A large task difference Δhg in the dispersion of the number of the SCRs of the emotional sweating means that the difference in the dispersion of the number of the SCRs of the emotional sweating between the time of solving the high difficulty level questions and the time of solving the low difficulty level questions is large. It can be said that a user who has obtained such a result has a tendency in which, when solving the high difficulty level questions, the task difference in the dispersion of the number of the SCRs of the emotional sweating becomes larger as compared with other users. Meanwhile, a small task difference Δhg in the dispersion of the number of the SCRs of the emotional sweating means that the difference in the dispersion of the number of the SCRs of the emotional sweating between the time of solving the high difficulty level questions and the time of solving the low difficulty level questions is small. It can be said that a user who has obtained such a result has a tendency in which, as the difficulty level of the questions becomes high, the task difference in the dispersion of the number of the SCRs of the emotional sweating becomes smaller as compared with other users.

It is appreciated from FIG. 41 that, when the task difference Δhg in the dispersion of the number of the SCRs of the emotional sweating is large, the accuracy rate R for questions becomes high, and that, when the task difference Δhg in the dispersion of the number of the SCRs of the emotional sweating is small, the accuracy rate R for questions becomes small. It is appreciated from the above that a person who has large dispersion of the number of the SCRs of the emotional sweating even for difficult questions tends to have a high accuracy rate R (i.e., be able to answer accurately even for difficult questions to the same degree as for simple questions). Conversely, it is appreciated that a person who has small dispersion of the number of the SCRs of the emotional sweating for difficult questions tends to have a low accuracy rate R (i.e., the accuracy rate for the difficult questions is lowered).

Herein, as described above, it can be seen from FIG. 28 that, when the accuracy rate is high, the arousal level is low, and when the accuracy rate is low, the arousal level is high. It can be inferred from the above that, when the task difference Δhg in the dispersion of the number of the SCRs of the emotional sweating is small, the arousal level of the user is lower than the predetermined standard. In addition, it can be inferred that, when the task difference Δhg in the dispersion of the number of the SCRs of the emotional sweating is large in the negative direction, the arousal level of the user is higher than the predetermined standard.

It is appreciated from the above that using the task difference Δhg in the dispersion of the number of the SCRs of the emotional sweating and the regression formulae in FIGS. 28 and 41 makes it possible to derive the arousal level of the user.

FIG. 42 illustrates an example of a relationship between a task difference Δhh [ms²/Hz] and the accuracy rate R [%]. The task difference Δhh [ms²/Hz] is a task difference in the number of the SCRs of the emotional sweating between the time of solving the high difficulty level questions and the time of solving the low difficulty level questions. The accuracy rate R [%] is the accuracy rate at the time of solving the high difficulty level questions. The task difference Δhh is a vector volume obtained by subtracting the number of the SCRs of the emotional sweating at the time of solving the low difficulty level questions from the number of SCRs of the emotional sweating at the time of solving the high difficulty level questions. In FIG. 42, data for respective users are plotted, and features of the entirety of users are represented by a regression formula (regression line). In FIG. 42, the regression formula is represented by R=a17×Δhh+b17.

A large task difference Δhh in the number of the SCRs of the emotional sweating means that the difference in the number of the SCRs of the emotional sweating between the time of solving the high difficulty level questions and the time of solving the low difficulty level questions is large. It can be said that a user who has obtained such a result has a tendency in which, when solving the high difficulty level questions, the task difference in the number of the SCRs of the emotional sweating becomes larger as compared with other users. Meanwhile, a small task difference Δhh in the number of the SCRs of the emotional sweating means that the difference in the number of the SCRs of the emotional sweating between the time of solving the high difficulty level questions and the time of solving the low difficulty level questions is small. It can be said that a user who has obtained such a result has a tendency in which, as the difficulty level of the questions becomes high, the task difference in the number of the SCRs of the emotional sweating becomes smaller as compared with other users.

It is appreciated from FIG. 42 that, when the task difference Δhh in the number of the SCRs of the emotional sweating is small, the accuracy rate R for questions becomes high, and that, when the task difference Δhh in the number of the SCRs of the emotional sweating is small, the accuracy rate R for questions becomes low. It is appreciated from the above that a person who has the large number of the SCRs of the emotional sweating even for difficult questions tends to have a high accuracy rate R (i.e., be able to answer accurately even for difficult questions to the same degree as for simple questions). Conversely, it is appreciated that a person who has the small number of the SCRs of the emotional sweating for difficult questions tends to have a low accuracy rate R (i.e., the accuracy rate for the difficult questions is lowered).

Herein, as described above, it can be seen from FIG. 28 that, when the accuracy rate is high, the arousal level is low, and when the accuracy rate is low, the arousal level is high. It can be inferred from the above that, when the task difference Δhh in the number of the SCRs of the emotional sweating is small, the arousal level of the user is lower than the predetermined standard. In addition, it can be inferred that, when the task difference Δhg in the number of the SCRs of the emotional sweating is large in the negative direction, the arousal level of the user is higher than the predetermined standard.

It is appreciated from the above that using the task difference Δhh in the number of the SCRs of the emotional sweating and the regression formulae in FIGS. 28 and 42 makes it possible to derive the arousal level of the user.

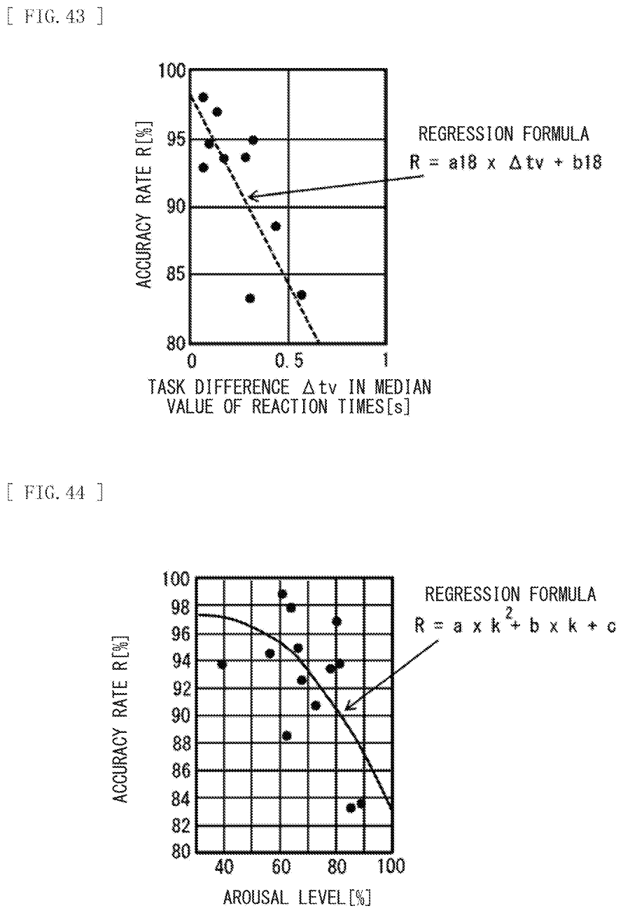

In addition, in the regression formula according to any of the first to fourth embodiments described above and the modification examples thereof, for example, as illustrated in FIG. 43, a task difference Δtv in a median value (median) of reaction times may be used in place of the task difference Δtv in the dispersion of the reaction times.

In addition, in the first to fourth embodiments described above and the modification examples thereof, the regression formula is not limited to a straight line (regression line), but may be a curve (regression curve), for example. The curve (regression curve) may be, for example, a quadratic function. The regression formula defining the relationship between the arousal level k [%] and the accuracy rate R [%] may be defined as a quadratic function (R=a×k2+bk+c), for example, as illustrated in FIG. 44.

In addition, the present disclosure may have the following configurations, for example.

(1)

A biological information processing device including:
a derivation section that derives emotional information about a target living body who is performing a specific task on the basis of at least one of biological information or action information acquired from the target living body; and
a classification section that classifies the emotional information acquired by the derivation section on the basis of a predetermined classification index.

(2)

The biological information processing device according to (1), further including an evaluation section that evaluates the target living body on the basis of a classification result by the classification section.

(3)

The biological information processing device according to (2), further including a storage section, in which
every time the derivation section acquires the emotional information, the classification section stores, in the storage section, the classification result by the classification section in association with an identifier of the target living body, and
the evaluation section selects a plurality of the identifiers suitable to constitute a specific group, on the basis of a plurality of the classification results stored in the storage section.

(4)

The biological information processing device according to any one of (1) to (3), further including a storage section, in which
the derivation section derives a feature amount corresponding to the classification index, on the basis of the emotional information, and stores, in the storage section, the derived feature amount in association with an identifier of the target living body.

(5)

The biological information processing device according to (4), further including an image data generator that generates image data in which the classification index and the feature amount are associated with each other.

(6)

The biological information processing device according to (4) or (5), in which the derivation section derives time series data as the emotional information, and stores, in the storage section, the derived time series data in association with the identifier of the target living body.

(7)

The biological information processing device according to (6), further including an image data generator that generates image data in which the classification index and the time series data are associated with each other.

(8)

The biological information processing device according to any one of (1) to (7), in which the biological information includes information about a brain wave, sweating, a heart rate, blood flow velocity, or a specific component contained in saliva.

(9)

The biological information processing device according to any one of (1) to (7), in which the action information includes information about facial expression, voice, or a reaction time.

(10)

The biological information processing device according to any one of (1) to (9), in which the emotional information includes at least one of an arousal level or comfort/discomfort of the target living body.

(11)

A biological information processing device including:
a storage section: and
a derivation section that derives emotional information about a target living body who is performing a specific task on the basis of at least one of biological information or action information acquired from the target living body, and stores, in the storage section, the derived emotional information in association with an identifier of the target living body.

(12)

The biological information processing device according to (11), in which
every time the derivation section derives the emotional information, the derivation section stores, in the storage section, the derived emotional information in association with the identifier of the target living body, and
the biological information processing device further includes an image data generator that generates image data in which pieces of the emotional information corresponding to a plurality of the identifiers are summarized in a mutually comparable manner.

(13)

The biological information processing device according to (12), further including:
a receiver that receives, from a user, selection of a plurality of pieces of the emotional information from among a plurality of pieces of the emotional information, or selection of a plurality of the identifiers from among a plurality of identifiers; and
a selector that selects a plurality of the identifiers suitable to constitute a specific group, on the basis of contents received by the receiver.

(14)

The biological information processing device according to (11), in which
every time the derivation section derives the emotional information, the derivation section stores, in the storage section, the derived emotional information in association with the identifier of the target living body, and
the biological information processing device further includes a selector that selects a plurality of the identifiers suitable to constitute a specific group, on the basis of a plurality of pieces of the emotional information stored in the storage section and a predetermined classification index.

(15)

The biological information processing device according to (12), in which
the derivation section derives time series data as the emotional information, and stores, in the storage section, the derived time series data in association with the identifier of the target living body, and
the image data generator generates, as the image data, image data in which pieces of the time series data corresponding to a plurality of the identifiers are aligned in time and superimposed on each other.

(16)

The biological information processing device according to any one of (11) to (15), in which the biological information includes information about a brain wave, sweating, a pulse wave, an electrocardiogram, a blood flow, a skin temperature, potential of facial muscles, eye potential, or a specific component contained in saliva.

(17)

The biological information processing device according to any one of (11) to (15), in which the action information includes information about facial expression, voice, blinking, breathing, or a reaction time for an action.

(18)

The biological information processing device according to any one of (11) to (17), in which the emotional information includes at least one of an arousal level or comfort/discomfort of the target living body.

(19)

A biological information processing system including:
an acquisition section that acquires at least one of biological information or action information from a target living body who is performing a specific task;
a derivation section that derives emotional information about the target living body on the basis of information acquired by the acquisition section; and
a classification section that classifies the emotional information acquired by the derivation section on the basis of a predetermined classification index.

(20)

A biological information processing system including:
a storage section;
an acquisition section that acquires at least one of biological information or action information from a target living body who is performing a specific task; and
a derivation section that derives emotional information about the target living body on the basis of information acquired by the acquisition section, and stores, in the storage section, the derived emotional information in association with an identifier of the target living body.

In a biological information processing device according to a first aspect of the present disclosure, and a biological information processing system according to a second aspect of the present disclosure, an arousal level is classified on the basis of a predetermined classification index. This makes it possible to classify a target living body with use of the arousal level that is objective data. As a result, for example, in a case of recruitment, it is possible to determine, from the arousal level of an applicant, whether or not the applicant is a desired talent. In addition, for example, in a case of determining project members, it is possible to determine, from the arousal levels of a large number of members, whether or not the members are suitable to constitute a specific group. It is therefore possible to reduce mismatches.

In a biological information processing device according to a third aspect of the present disclosure and a biological information processing system according to a fourth aspect of the present disclosure, a derived arousal level is stored, in a storage section, in association with an identifier of a target living body. This makes it possible to classify the target living body with use of the arousal level that is objective data. As a result, for example, in a case of recruitment, it is possible to determine, from the arousal level of an applicant, whether or not the applicant is a desired talent. In addition, for example, in a case of determining project members, it is possible to determine, from the arousal levels of a large number of members, whether or not the members are suitable to constitute a specific group. It is therefore possible to reduce mismatches.

This application claims the priority on the basis of Japanese Patent Application No. 2021-056031 filed on Mar. 29, 2021 and Japanese Patent Application No. 2021-132937 filed on Aug. 17, 2021 with Japan Patent Office, the entire contents of which are incorporated in this application by reference.

It should be understood by those skilled in the art that various modifications, combinations, sub-combinations, and alterations may occur depending on design requirements and other factors insofar as they are within the scope of the appended claims or the equivalents thereof.

1. A biological information processing device comprising:
a derivation section that derives emotional information about a target living body who is performing a specific task on a basis of at least one of biological information or action information acquired from the target living body; and
a classification section that classifies the emotional information acquired by the derivation section on a basis of a predetermined classification index.

2. The biological information processing device according to claim 1, further comprising an evaluation section that evaluates the target living body on a basis of a classification result by the classification section.

3. The biological information processing device according to claim 2, further comprising a storage section, wherein
every time the derivation section acquires the emotional information, the classification section stores, in the storage section, the classification result by the classification section in association with an identifier of the target living body, and
the evaluation section selects a plurality of the identifiers suitable to constitute a specific group, on a basis of a plurality of the classification results stored in the storage section.

4. The biological information processing device according to claim 1, further comprising a storage section, wherein
the derivation section derives a feature amount corresponding to the classification index, on a basis of the emotional information, and stores, in the storage section, the derived feature amount in association with an identifier of the target living body.

5. The biological information processing device according to claim 4, further comprising an image data generator that generates image data in which the classification index and the feature amount are associated with each other.

6. The biological information processing device according to claim 4, wherein the derivation section derives time series data as the emotional information, and stores, in the storage section, the derived time series data in association with the identifier of the target living body.

7. The biological information processing device according to claim 6, further comprising an image data generator that generates image data in which the classification index and the time series data are associated with each other.

8. The biological information processing device according to claim 1, wherein the biological information comprises information about a brain wave, sweating, a heart rate, blood flow velocity, or a specific component contained in saliva.

9. The biological information processing device according to claim 1, wherein the action information comprises information about facial expression, voice, or a reaction time.

10. The biological information processing device according to claim 1, wherein the emotional information comprises at least one of an arousal level or comfort/discomfort of the target living body.

11. A biological information processing device comprising:
a storage section: and
a derivation section that derives emotional information about a target living body who is performing a specific task on a basis of at least one of biological information or action information acquired from the target living body, and stores, in the storage section, the derived emotional information in association with an identifier of the target living body.

12. The biological information processing device according to claim 11, wherein
every time the derivation section derives the emotional information, the derivation section stores, in the storage section, the derived emotional information in association with the identifier of the target living body, and
the biological information processing device further comprises an image data generator that generates image data in which pieces of the emotional information corresponding to a plurality of the identifiers are summarized in a mutually comparable manner.

13. The biological information processing device according to claim 12, further comprising:
a receiver that receives, from a user, selection of a plurality of pieces of the emotional information from among a plurality of pieces of the emotional information, or selection of a plurality of the identifiers from among a plurality of identifiers; and
a selector that selects a plurality of the identifiers suitable to constitute a specific group, on a basis of contents received by the receiver.

14. The biological information processing device according to claim 11, wherein
every time the derivation section derives the emotional information, the derivation section stores, in the storage section, the derived emotional information in association with the identifier of the target living body, and
the biological information processing device further comprises a selector that selects a plurality of the identifiers suitable to constitute a specific group, on a basis of a plurality of pieces of the emotional information stored in the storage section and a predetermined classification index.

15. The biological information processing device according to claim 12, wherein
the derivation section derives time series data as the emotional information, and stores, in the storage section, the derived time series data in association with the identifier of the target living body, and
the image data generator generates, as the image data, image data in which pieces of the time series data corresponding to a plurality of the identifiers are aligned in time and superimposed on each other.

16. The biological information processing device according to claim 11, wherein the biological information comprises information about a brain wave, sweating, a pulse wave, an electrocardiogram, a blood flow, a skin temperature, potential of facial muscles, eye potential, or a specific component contained in saliva.

17. The biological information processing device according to claim 11, wherein the action information comprises information about facial expression, voice, blinking, breathing, or a reaction time for an action.

18. The biological information processing device according to claim 11, wherein the emotional information comprises at least one of an arousal level or comfort/discomfort of the target living body.

19. A biological information processing system comprising:
an acquisition section that acquires at least one of biological information or action information from a target living body who is performing a specific task;
a derivation section that derives emotional information about the target living body on a basis of information acquired by the acquisition section; and
a classification section that classifies the emotional information acquired by the derivation section on a basis of a predetermined classification index.

20. A biological information processing system comprising:
a storage section;
an acquisition section that acquires at least one of biological information or action information from a target living body who is performing a specific task; and
a derivation section that derives emotional information about the target living body on a basis of information acquired by the acquisition section, and stores, in the storage section, the derived emotional information in association with an identifier of the target living body.
